(19) United States
(12) Patent Application Publication
(10) Pub. No.: US 2005/0165480 A1
(43) Pub. Date: Jul. 28, 2005

(54) Endovascular treatment devices and methods

(76) Inventors: Maybelle Jordan, Potomac, MD (US); Arindam Datta, Hillsboro, NJ (US); Craig F. Friedman, Westport, CT (US); George Sanderson, Clark, NJ (US); John Kula, Birdsboro, PA (US)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 10/900,982

(22) Filed: Jul. 27, 2004

Related U.S. Application Data
(60) Provisional application No. 60/538,597, filed on Jan. 23, 2004.

(51) Int. Cl. A61F 2/20

(52) U.S. Cl. 623/9

(57) ABSTRACT

A device for treating or preventing a vascular condition at a mammalian vascular site, comprises an implant formed from a compressible, reticulated elastomeric matrix in a shape conducive to delivery through a delivery instrument. One or more implants are delivered in a compressed state to the mammmalian vascular site where each implant recovers substantially to its uncompressed state following deployment from a delivery instrument. In a preferred embodiment the matrix comprises cross-linked polycarbonate polyurethane-urea or cross-linked polycarbonate polyurea-urethane. In another preferred embodiment the matrix comprises a cross-linked polycarbonate polyurethane. In a yet further embodiment, the matrix comprises thermoplastic polycarbonate polyurethane or thermoplastic polycarbonate polyurethane-urea.

### CROSS-REFERENCE TO RELATED APPLICATION

This application is based upon co-pending, commonly assigned, U.S. provisional patent application Ser. No. 60/538,597, filed Jan. 23, 2004, incorporated herein by reference.

### FIELD OF THE INVENTION

The present invention relates to endovascular treatment devices and methods useful for treatment of vascular conditions such as vascular aneurysms and other vascular abnormalities, defects or malformations. In particular, although not exclusively, the invention relates to devices and methods useful in conjunction with grafts or graft implantation procedures, for example, aneurysm endografts and aneurysm endograft implantation procedures, which devices and methods are helpful in providing management of leakage commonly associated with such endografts.

### BACKGROUND OF THE INVENTION

An abdominal aortic aneurysm (hereinafter “AAA”) is a common clinical problem which occurs when the walls of the descending aorta weaken and bulge into a sac. The aortic artery descends from the heart to the abdominal area where it bifurcates into the right and left common iliac arteries. Each common iliac artery in turn bifurcates into the internal iliac and femoral arteries, which supply blood to one of the legs. Over time, a weakened artery that is normally about 2.5 cm in diameter can expand to 5.0 cm or more in diameter. An AAA is often recognized by the art to exist when an area of the aortic wall has expanded to generally more than 1.5 times its normal vessel diameter.

As of 2003, approximately 200,000 new cases of AAA are diagnosed in the U.S. each year. AAAs are the 13th leading cause of death in the U.S. and are responsible for approximately 20,000 deaths per year. AAAs primarily affect the elderly, and their incidence increases with age, affecting up to 10% of men over the age of 80 years.

The condition is usually asymptomatic and is frequently detected during physical exam or as an incidental finding to X-ray, CT or MRI studies. A primary objective in the treatment of AAAs is to prevent death from rupture. Once an asymptomatic AAA is discovered, the question becomes the probability of rupture. Rupture risk increases with the size of the aneurysm: rupture rates are 25-40% at 5 years for aneurysms greater than 5 cm in diameter, 5-7% at 5 years for aneurysms 3.5-5.0 cm in diameter, and approaching 0% at 5 years for those aneurysms less than 3.5 cm.

When an aortic aneurysm bursts, the patient bleeds into the internal body cavity and the event is usually fatal within minutes. Only 10-15% of patients survive a ruptured AAA. Moreover, the odds of surviving emergency surgery to repair a ruptured aneurysm are low; only 50% of patients survive an emergency repair procedure.

Conventional treatment for AAAs involves an invasive open surgical procedure in which the patient's chest is opened and a tubular graft is placed or sewn into the aneurysm space. Once the graft is sewn into place, the patient's blood flows through the newly created synthetic channel or vessel. The graft is intended to reduce and/or eliminate pressure build-up and reduce and/or eliminate flow into the perigraft space between the graft and the aneurysmal vessel wall, thereby reducing the risk of AAA rupture.

Catheter-delivered endovascular grafts also known as “endografts”, or stents, have been employed as a minimally invasive alternative to open surgical repair of AAAs since the introduction of the first endografts by commercial suppliers such as Guidant and Medtronic in the U.S. in 1999. Today, there are a number of commercial companies offering and/or developing endovascular grafts, including Medtronic (AneuRx, Talent), W. L. Gore (Excluder), Cook (Zenith), Boston Scientific/TriVascular (TriVascular), and Endologix (PowerLink). Endografts typically comprise a tubular metallic frame, flexible fabric such as ePTFE or polyester covering the frame, and anchoring components such as hooks, barbs, or clips to secure the graft to the vessel wall. Endografts can be implanted using a catheter which is introduced into the vascular system through an incision in the femoral artery in the leg. The endograft forms a synthetic channel through the aneurysm sac that is intended to isolate the aneurysm from the hemodynamic forces and pressures of the vascular system.

A problem occurring with many endovascular grafts is that of residual flow into the perigraft space between the endograft and the aneurysmal vessel wall, a complication commonly referred to as an “endoleak”. The persistence of pressure and/or reintroduction of pressure on the aneurysm walls can place the patient at continued risk of rupture, particularly when the endoleak is accompanied by an increase in aneurysm size. Various studies and registries have reported that 20% to 40% of patients undergoing endovascular repair (EVR) experience an endoleak at some point after endograft deployment.

There are four types of endoleaks. Type I endoleaks are device-related leaks that result from a failure to adequately seal the attachment sites of the endograft to the vessel walls. These leaks are aggressively treated during the endograft procedure. Type II endoleaks are leaks caused by retrograde flow from collateral arteries such as the lumbar arteries or the inferior mesenteric artery into the sac. Previously there was no satisfactory treatment approach to combat Type I or Type II endoleaks. Type III endoleaks are leaks arising from one or more defects in the graft itself, such as a hole in the fabric or a disjointed connection between modular components of the endograft, which leaks manifest themselves post-operatively. Type III leaks are also device-related and aggressively treated as soon as they are detected. Type IV endoleaks are leaks caused by fabric porosity and typically subside within about 30 days.

The art lacks a fully satisfactory and effective approach to treatment of endoleaks, and applicants are not aware of any acceptable device approved by the U.S. Food and Drug Administration (“FDA”) to address this problem. Some proposed treatment methods include aggressively treating Type I or Type II endoleaks using metallic embolization coils. However, this approach has not been effective in resolving or treating endoleaks on a consistent basis.

The treatment of any type of vascular malformation such as endoleaks or aneurysm space is very challenging owing to difficulty in accessing the target space especially in the presence of existing endografts or endografts placed in the aneurysm sac during the surgery. In addition, the difficulty in delivering large devices, preferably in a compressed state and pushed through the entire length of the delivery catheters, raises issues and challenges that have not been addressed by prior art or existing devices.

Known secondary procedures to seal off endoleaks are technically demanding and are not always successful in creating a durable exclusion of perigraft flow. These procedures include transarterial embolization of feeding and draining vessels using coils, and direct puncture and injection of thrombin and/or coils into the aneurysm sac itself.

Transarterial embolization of feeding and draining vessels is a technically demanding and time-consuming procedure, and it does not always lead to complete endoleak occlusion, as new collateral vessels often emerge and continue to perfuse the sac. Direct puncture and injection of thrombin and/or coils into the sac is also a less-than-ideal solution, due to the significant risks of embolization through the draining vessels, the costs associated with use of large numbers of platinum coils, and the difficulty of targeted positioning of one or more coils at the endoleak nexus within the sac. It is also well known that the use of coil is frequently associated with recanalization of the site leading to full or partial reversing of the endoleak occlusion.

Several methods have been proposed for addressing the problem of endoleaks, but they all have certain drawbacks and none is entirely satisfactory and effective for treating or preventing endoleaks. There are several difficult challenges and issues associated with procedures, methods and delivery methods for satisfactory and effective for treatment or prevention of endoleaks and the current procedures do not fully appreciate the complexities and difficulties associated with accessing the vascular malfunction sites surrounding the endografts. Thus, there is a need for an effective method and device for treating and/or preventing endoleaks.

Further, there are many clinical situations that require therapeautic embolization, including vessel occlusion (e.g., internal iliac artery embolization, inferior mesenteric artery embolization, lumbar artery embolization, and renal artery embolization); arteriovenuous malformations; arteriovenuous fistulas; psuedoaneurysms, gastrointestinal hemorrhage; and bleeding due to tumors or trauma. Most contemporary vascular occlusion devices, such as coils, thrombin, glue, GELFOAM, PVA articles, alcohol injections, etc., have serious limitations or drawbacks, including, but not limited to, early or late recanalization, incorrect placement or positioning, and migration. Also, some of the devices are physiologically unacceptable and engender unacceptable foreign body reactions or rejection. Accordingly, there is a clinical need for an embolization agent that produces permament biological occlusion, can be delivered to a target vascular or other site with minimal risk of migration, is sufficiently large to reduce the number of implants and reduce surgery time but can still be delivered in a compressed state through small diameter catheters and is substantially physiologically acceptable.

### OBJECTS OF THE INVENTION

It is an object of the invention to provide endovascular treatment devices and methods useful for treatment of vascular conditions such as vascular aneurysms and other vascular abnormalities, defects, or malformations.

It is also an object of the invention to provide devices or implants and methods useful in conjunction with grafts or graft implantation procedures, for example, aneurysm endografts and aneurysm endograft implantation procedures, which devices and methods are helpful in providing management of leakage commonly associated with such endografts.

It is a further object of the invention to provide new devices that can solve the problem of treating and preventing leakage to and from endovascular grafts employed to manage or control vascular defects or abnormalities, for example, aneurysms, with a low risk of embolization.

It is a yet further object of the invention to provide new devices that can solve the problem of treating and/or preventing leakage of other more general embolization applications, including the treatment of arteriovenous fistulas, arteriovenous malformations, arterial or venous embolizations, vessel wall perforations, or other such defects or abnormalities as may be appropriate, whether or not such problems are strictly describable as endoleaks.

It is a yet further object of the invention to provide a device, implant, or apparatus for controlling leakage into aneurysm perigraft spaces that can be attributed to backflow through microvasculature vessels feeding into or draining from the aneurysm.

It is a yet further object of the invention to provide endovascular treatment devices and methods utilizing arterially deliverable implants that are resistant to recanalization and migration.

It is a yet further object of the invention to provide implants or devices that are biodurable and support tissue ingrowth/endothielization.

It is a further object of the invention to provide vascular occlusion devices comprising reticulated, resilient, polyurethane foam implants.

It is a further object of the invention to provide single or a few number of sufficiently large implants to reduce or minimize the number of implants and reduce surgery time but can still be delivered in a compressed state through the small diameter catheters and deliver them through tortuous channels to access difficult target sites.

It is a further object of the invention to provide systems to deliver endovascular treatment devices through tortuous channels to access target sites.

These and other objects of the invention will become more apparent from the discussion below.

### SUMMARY OF THE INVENTION

The present invention solves a problem, namely, the problem of providing endovascular treatment devices and methods that can provide post-operative or prophylactic or peri-operative treatments for endovascular problems that threaten the integrity of the vasculature. The endovascular treatment devices and methods provide a low risk of embolization, can be easily effected, and are efficient.

According to the invention, new devices and methods are provided that can solve the problem of treating and preventing leakage from endovascular grafts employed to manage or control vascular defects or abnormalities, for example, aneurysms, with a low or minimal risk of embolization. A device, or apparatus, or method is provided for controlling leakage into an aneurysm perigraft space, that is, the space surrounding and contiguous with an endograft within an artery or other vasculature, that can be attributed to backflow through microvasculature vessels feeding into or draining from the aneurysm.

According to the invention endovascular treatment devices and methods utilizing arterially deliverable implants are provided that are resistant to recanalization and migration. Arterial delivery via a catheter, or other introducer, is a relatively low-trauma procedure which can be employed post-operatively to address complications of more invasive measures such as the surgical implantation of vascular grafts and also, in the case of catheter-delivered endovascular grafts that are an minimally invasive, is an alternative to open surgical repair. It will be understood that in most cases, implants designed for arterial delivery can, if desired, be delivered percutaneously, for example, as an adjunct to a more substantial surgical procedure.

In one aspect, the invention solves these problems by providing a device or method for the treatment or prevention of endoleaks, for example, an aneurysm surrounding an implanted endovascular graft, the device or method comprising delivering a plurality of reticulated, fluid-pervious elastomeric implants in a compressed state, into the target site and which recover partially or substantially on release from the delivery system. More particularly, the implants target vascular embolization of endoleak nexus inside the sac volume. The inventive implantable device is reticulated, i.e., comprises an interconnected and intercommunicating network of pores and/or voids that provides fluid permeability throughout the implantable device and permits cellular ingrowth and proliferation into the interior of the implantable device.

In one embodiment, the invention solves these problems by providing a method for the treatment or prevention of endoleaks from an implanted endovascular graft, the method comprising delivering, in a compressed state, a plurality of fluid-pervious elastomeric implants, formed of a biodurable reticulated polyurethane matrix, to a perigraft target site being a volume contiguous with and external to the endovascular graft, wherein each delivered implant has a bulk volume in a relaxed state prior to compression which is substantially less than the actual or apparent volume of the target site so that a plurality of implants can readily be accommodated in the target site.

Some embodiments of the invention comprise a method or procedure wherein a group of fluid-pervious elastomeric reticulated biodurable implants is introduced into a target site, for example, via catheter, needle, or cannula, to fill or at least substantially fill the perigraft space between an endograft and an aneurysm wall. Such a procedure can be effective to limit or seal off endoleaks from within the aneurysm sac, and may also prevent the occurrence of future endoleaks. Such a procedure may also stabilize the aneurysm sac, and has the potential to provide support to the endograft and prevent future migration of the graft.

Embodiments of the invention include delivering reticulated elastomeric implants to a target site and releasing the implants into the target site with the location and orientation of each individual implant being determined by the local anatomy, by an endograft, if employed, and by neighboring implants. Thus, the location and orientation of a particular implant, or any implant, may not be predetermined, but may be passively determined by the implant according to the environment into which it is introduced. In general, but without excluding the possibility, the implants employed in the invention do not need to be actively secured or attached to any ambient structure at the target site. However, it is contemplated that some embodiments of the invention will sufficiently fill or pack the target site with implants that most, if not all, the implants will be held in position by their neighbors, the site anatomy, or an endograft or other prosthetic. Advantageously the implants can be formed of a biodurable material to promote permanent sac occlusion and endoleak resolution or treatment of other vascular malfunctions or irregularities.

The endovascular graft can be annular, or partially annular, defining a space for the passage of bodily fluid, notably blood, internally through the graft. As is well known in the art, the endograft can be tubular or may comprise a Y-shaped tube providing one or more passageways for arterial blood flow to bypass a damaged or defective vascular region.

In another embodiment, the invention provides devices and methods for occupying a target biological site with transarterially deliverable implants that are expandable in situ and recover partially or substantially or fully to its original volume and are resistant to migration. The implant material and structure are preferably selected to resist migration of the implants out of the target site in the long-term by employing materials and structure that permit or encourage tissue ingrowth and proliferation into the implant interiors so that it becomes bio-integrated to the target site. To resist migration in the short term, the implants can usefully have migration-inhibiting dimensions upon arrival in the target site or assume such dimensions shortly thereafter and prior to possible migration of the implant out of the target site.

In another aspect the invention solves these problems by providing a device or method for the treatment or prevention of endoleaks leading into or draining into a target vascular site such as an aneurysm, the device or method comprising delivering a single or plurality of reticulated, fluid-pervious elastomeric implants in a compressed state, into the target site and which recover partially or substantially on release from the delivery system. In another embodiment, implants are delivered transarterially to embolize or occlude feeder or draining vessels that bring in addition fluid or blood into the aneurysm, e.g., endoleaks arising from the internal iliac artery in aorta-iliac aneurysm.

Advantageously the implants are elastomeric and have inherent resilient expansion properties, when compressed they exert an expansive stress on the compressing device to increase their volume promptly after or during their release from the introducer or delivery device. The compressing device may be a catheter, needle, cannula, or other introducer or a loading device employed to load the implants one or more at a time into the introducer. Either the implant quickly expanding to a volume selected to be incapable of migration or the press of surrounding structures, including, possibly, other implants, prevents both expansion and migration.

Preferably the implants are fabricated of at least partially hydrophobic elastomeric material. The implant material optionally may have a hydrophilic surface treatment or hydrophilic coating for any desired purpose, for example, to facilitate delivery of a biologically active substance which may be attached to the hydrophilic surface or coating. However, the invention includes many useful embodiments that lack such a hydrophilic surface or coating and present hydrophobic surfaces to their environment.

Useful embodiments of implant can be fabricated of biocompatible materials which do not readily induce adverse biological reactions or release biologically harmful substances, a foreign body reaction being regarded as desirable in the context of the invention. Preferably materials are employed that are biodurable, being resistant over time to breakdown when continuously exposed to a biological environment. The invention includes embodiments employing materials that are both biocompatible and biodurable.

Being biodurable, advantageously the implants are capable of maintaining their mechanical and chemical structural integrity, in situ, over time, for example, until substantially ingrown with tissue, for the intended life of the implant, or for the expected life of the host organism. Some useful embodiments of the invention employ implants that are not bioabsorbable, do not break down into or liberate fragments or particles in situ that could provide a risk of migration and undesired embolization, and which can be expected to become mechanically secured in situ, preventing migration, by natural biological processes.

The implant matrix microstructure is preferably reticulated or substantially reticulated and may comprise interconnected and intercommunicating networks of pores and/or voids, either by being formed having a reticulated structure and/or undergoing a reticulation process. The network comprises open inter-connected cells of appropriate pore or cell size to facilitate tissue ingrowth and proliferation and subsequent bio-integration. Where the cell walls between adjacent cells are least partially removed by reticulation or may have been subject to a reticulation process step to remove cell walls, adjacent reticulated cells open into, are interconnected with, and communicate with each other. In one embodiment, there are few, if any, “window panes” separating adjacent cells. Such structure can be provided by one or more interior networks of passages, open cells, pores or other volumes that communicate each with its neighbors to permit fluid flow through the individual implant and permits cellular ingrowth and proliferation into the interior of the implantable device. Advantageously the implant matrix is resistant to biological degradation and non-resorbable. In one embodiment of the invention, the matrix is or comprises reticulated, elastomeric, biodurable polycarbonate polyurethane material.

Some useful implants for employment in the invention offer controlled resistance to blood flow in situ at a target site, without being significantly dislodged or caused to migrate by the blood flow.

Thus, for example, suitable implants can resist blood flow in or through the aneurysm while remaining usefully positioned in the aneurysm. When the reticulated elastomeric implants are placed in or carried to a conduit or a vessel through which body fluid passes or accumulates such as the targeted aneurysm sac or side branch or feeder and/or drainer vessels, it will provide an immediate resistance to the flow of body fluid such as blood. This will be associated with an inflammatory response and the activation of a coagulation cascade leading to formation of a clot, owing to a thrombotic response. Thus, local turbulence and stagnation points induced by the implantable device surface may lead to platelet activation, coagulation, thrombin formation and clotting of blood. The desirable natural processes of thrombosis which will help control the aneurysm may be induced.

Preferably the individual implants have morphologies to accommodate fibrotic cellular ingrowth. It is also preferable that the implant matrix material have a microstructure intended to promote cellular proliferation and tissue ingrowth into, and preferably throughout the interior of the implant. Optionally, the implants may be thrombogenic. Such tissue ingrowth coupled with natural processes of foreign body thrombosis can stabilize the aneurysm and secure the plurality or group of implants and the endograft in position. Over time, this induced fibrovascular entity resulting from tissue ingrowth can cause the implantable device to be incorporated into the conduit. It may also prevent recanalization of the conduit. To this end the implant matrix microstructure needs to be accessible to liquids, and is preferably accessible to bodily fluids, including blood, which is somewhat viscous.

Unlike known solutions that attempt space filling with a swellable material, it is believed clinically desirable pursuant to the invention, not only to occlude the targeted vascular space, but also to engender tissue ingrowth into the target volume to create a durable fibrosis that will serve to seal the endoleakage, stabilize the aneurysm sac, provide support to the endograft, and mitigate the risk of device migration which may be associated with endografts and prior attempts to control endoleaks employing absorbable gels or the like. Tissue ingrowth can lead to incorporation and integration with the body lumen or surrounding vessels or tissues and very effective resistance to migration of the implantable device and re-canalization over time

In another embodiment, the invention provides apparatus for compressing and delivering the implants to a target vascular site. Preferably the delivery instrument can hold the implants in a compressed state for delivery and transport them preferably percutaneously without large frictional resistance, and can release the compressed implants to eventually expand at the target site on delivery. Thus, the delivery apparatus can comprise one or more implant packing members to hold the implants individually or as a group of two or more, in a compressed state during transport from an extracorporeal location through the patient's body, traversing the tissues, or vasculature or both, to the target site. A suitable delivery instrument can also comprise a release member, operable by the surgeon or other user to release the transported implant or implants at or near the target site. It also addresses the issue of accessibility of the endoleaks or the vascular malfunction sites especially those difficult to access area surrounding the endografts.

The invention provides simple and potentially effective treatments for a wide range of vascular disorders, which, if natural processes of thrombosis and cellular ingrowth occur in the manner contemplated herein, consistently with the animal studies described herein, offers the potential for uniquely effective embolization treatments, which are adjunctive to AAA endograft procedures. Furthermore, the invention offers potential means for both treating and preventing endoleakage at a target vascular site.

In another embodiment, a single or a few number of sufficiently large implants to reduce or minimize the number of implants and reduce surgery time can still be delivered in a compressed state through small diameter catheters and can be delivered through tortuous channels to access difficult target sites.

Employment of a considerable number, for example, a group of from about 1 to about 100, or even about 30 or more, fluid-pervious elastomeric implants that are relatively small compared with the target site can be advantageous in facilitating desirable filling of the anisotropic sac geometry of a typical AAA or other problematic vascular site. This is necessitated by the extreme difficulty and formidable challenge in delivering a few large implants through a long narrow or small diameter catheter. The endoleak treatment sites are at times made even more difficult to access owing to narrow passage and lack of maneuverability in the space surrounding the pre-existing endograft or the endograft that is put in prior to the implants being inserted for prophylactic or peri-operative treatments for endovascular problems. Also, it will be easier to fill or substantially fill the aneurysm sac with smaller implants given the anisotropic irregular size and shape of the aneurysm sac. Due to use of such a group of small, low density, compressible implants good accommodation of the implanted matrix to the geometry of an anisotropic or other target site may be obtained. In certain cases with discrete, localized endoleaks that can be precisely located and accessed, it is possible that a targeted number of implants can be used to embolize the nexus of the endoleak or leaks. The targeted number of implants may be relatively small, for example, from about 1 to about 10, preferably from about 2 to about 8, and may not completely fill nor obliterate the sac or the vessel. It is contemplated that, with the passage of time, tissue ingrowth, responsive to the particular morphology of the implants may help to fill with tissue volumes of the target site that are not occupied by implant material. In another embodiment, targeted number of implants may completely fill and obliterate the sac.

Another embodiment of the invention relates to the use of reticulated, resilient, polyurethane foam implants delivered in a compressed state for vascular occlusion. The preferred material comprises cross-linked polycarbonate polyurea-urethane material, which offers the critical characteristics for a percutaneously delivered endovascular implant, namely, reticulated structure, pore size, resilient recovery, compression set, and flow-through.

### BRIEF DESCRIPTION OF THE DRAWINGS

FIG. 1 is a schematic sectional view of the abdominal region of a descending human aorta bearing a well-developed aneurysm which has been treated with an endograft, wherein the perigraft space around the endograft is filled with porous elastomeric implants in accordance with method and device embodiments of the invention;

FIG. 2 illustrates a hollow cylindrical embodiment of reticulated elastomeric implant suitable for employment in the methods or useful as components of the devices of the embodiments of the invention described with reference to FIG. 1;

FIG. 3 is a view similar to FIG. 2 of a hollow bullet-shaped implant;

FIG. 4 is a view similar to FIG. 2 of a hollow frustoconical-shaped implant;

FIG. 5 is a view similar to FIG. 1 of another endograft-bypassed abdominal aortic aneurysm that can be treated by the methods and devices of the invention, showing the extension of the aneurysm along one common iliac and one method for occluding a branch artery;

FIG. 6 is a schematic view of an implant emerging from a catheter at a target site in a host animal pursuant to the practice of a method of the invention;

FIG. 7 is a perspective view of a loader apparatus useful according to the invention;

FIG. 8 is a partly cross-sectional view of the loader apparatus shown in FIG. 7;

FIG. 9 is a partly cross-sectional view of a split delivery catheter useful according to the invention;

FIG. 10 is a cross-sectional view across line 10-10 of the catheter shown in FIG. 9;

FIG. 11 is a lateral view of an obdurator or pusher useful according to the invention;

FIGS. 12 and 13 are each a cross-sectional view of the distal end of an implant delivery catheter showing deployment of the implant using an obturator;

FIGS. 14 to 16 are each a micrograph showing the biological tissue response to the implant of the invention placed in a rabbit carotid artery for one month;

FIGS. 17A and 17B represent cross-sectional views of a foam implant and a stainless steel coil, respectively, in the external iliac artery of a pig at one week; and

FIG. 18 represents a 20× magnification cross-sectional view of the left iliac artery showing cellular infiltration into the struts of the foam implant with minimal inflammatory response, swine peripheral model at one week sacrifice.

### DETAILED DESCRIPTION OF THE INVENTION

Endoleak treatment aspects of the invention will now be described by way of illustrative examples of the practice of the invention as applied to the treatment of AAA endoleaks. It is to be understood that the described devices, apparatus and methods can be usefully employed to treat a wide range of vascular conditions, additional to AAA endoleaks, with or without modification, including vascular aneurysms and other vascular abnormalities, defects, or malformations, as disclosed herein or as will be apparent to those skilled in the art. Such other aneurysms can include other aortic aneurysms, aneurysms of the iliac, femoral, popliteal, sub-clavian arteries or visceral arteries, the latter including the renal and mesenteric arteries, as well as aneurysms of the thoracic segment of the aorta.

As stated above, the methods and devices of the present invention are useful, inter alia, for treating endoleaks associated with endografts. The terms “endograft” and “endoleak” are used herein in a manner recognized in the art to connote, respectively, an endovascular graft and a leak from or in the vicinity of an endovascular graft. It will be understood that endovascular grafts usually, but not always, have an annular or tubular configuration and that endoleaks are usually, but not always, outward leaks from within the anatomical vessel past the endograft into the perigraft space around the graft.

The inventive devices and methods can also be employed to treat leakage associated with a stent, a tubular graft, a stent-graft, a coated stent, a covered stent, an intravascular flow modifier, or other endovascular implant device whether or not such devices are strictly describable as “endografts”, which leakage may place a patient at risk for aneurysm rupture. Additionally, the devices and methods can be used for other embolization applications, including the treatment of arterio-venous fistula, arterio-venous malformation, arterial embolizations, vessel wall perforation or, other such defect or abnormality as may be appropriate, whether or not such problems are strictly describable as endoleaks. Suitable such applications, and others, will be apparent to those skilled in the art based on the disclosure herein.

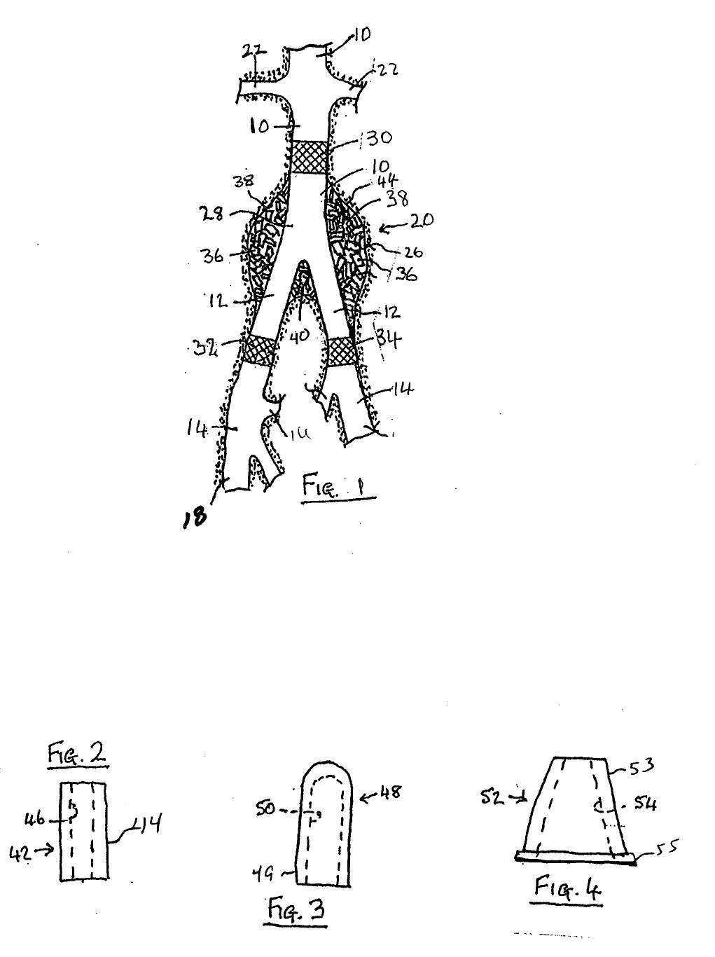

As shown in FIGS. 1 and 5, the illustrated descending aorta 10 bifurcates downwardly to form the common iliac arteries 12 which in turn each divide into an external iliac artery 14 and an internal iliac artery 16. External iliac artery 14 eventually becomes the femoral artery 18. As shown, an aortic aneurysm 20 has developed in the vicinity of the bifurcation of aorta 10 into the common iliac arteries 12. Upwardly of the iliac arteries 12, 14, 16, the renal arteries 22 branch laterally from the aorta 10 and lead to the kidneys 24 (as shown in FIG. 5). The aortic aneurysm 20 has a distended aneurysm wall 26 and occupies a substantial portion of aorta 10, from just beneath the renal arteries 22 to a short distance past the point of bifurcation of the aorta 10 into the common iliac arteries 12.

A “trouser”, or Y-shaped, endograft 28, sometimes called a stent, has an upper end 30 and two lower ends 32, 34. Each end 30, 32, 34, respectively, is secured in known manner to the aorta 10 and to the common iliac arteries 12, respectively. A primary function of endograft 28 is to bypass aneurysm 20, carrying the arterial blood flow from aorta 10 to common iliac arteries 12 and reducing the pressure on aneurysm wall 26, thereby preventing or reducing its chances of rupture or failure.

Many forms of suitable endograft 28 are known to those skilled in the art, for example, as described in the references cited hereinabove, and may be employed for the purposes of the present invention. Also possibly useful in the practice of the invention are devices and methods such as, and including, but not limited to, a number of commercial companies offering and/or developing endovascular grafts, including Medtronic (AneuRx, Talent), W.L. Gore (Excluder), Cook (Zenith), Boston Scientific/-TriVascular (TriVascular), and Endologix (PowerLink).

Some known endografts that may be employed comprise a tubular metallic frame, covered with a flexible fabric membrane formed of a suitable material such as ePTFE or polyester, and having anchoring components such as hooks, barbs, or clips to secure the graft to the vessel wall. The methods and devices of the present invention are believed effective with a wide range of types of known endografts and to be potentially useful with many endograft structures that will be devised in the future.

One of the major issues not addressed by the endovascular grafts is the problem of residual flow into the perigraft space between the endograft and the aneurysmal vessel wall, a complication commonly referred to as endoleaks. The sources of leaks vary from device-related issues during the procedure and retrograde flow from collateral arteries such as the lumbar arteries or the inferior mesenteric artery into the sac to leaks arising from a defect in the graft itself, such as a hole in the fabric or a disjointed connection between modular components of the endograft and undesired fabric porosity. The persistence of pressure and/or reintroduction of pressure or pressure build-up on the aneurysm walls can place the patient at continued risk of rupture, in particular when the endoleak is accompanied by an increase in aneurysm size.

As described above, aneurysm bypass endografts such as endograft 28 are subject to leakage. The aneurysm treatment thus can be made significantly more effective over just placing an endovascular graft in the aneurysm by additionally filling the perigraft space between the endograft and the aneurysm wall to seal off endoleak(s) from within the aneurysm sac and prevent the occurrence of future endoleaks and thus stabilize the aneurysm sac. These can be achieved by packing the aneurysm sac, embolizing the endoleak nexus within the sac, and occluding the feeder vessels such as collateral arteries that drain or bring additional fluid or blood into the sac.

With a view to managing endoleaks, the methods and devices of this aspect of the present invention provide a group or plurality of relatively small elastomeric, at least partially reticulated implants 36 disposed within what, for delivery purposes, may be described as a target site, aneurysm volume 38, being, in this case, the available volume within aneurysm 20 around endograft 28, also known as the perigraft space. Reticulated structure comprises of a morphology in which the pores of the foam are inter-connected with a continuous passage throughout the entire volume of the implant. Alternatively, a group of implants comprising a small number of larger at least partially reticulated elastomeric implants 36 of standardized shape or shapes selected to fit the target site collectively, may be employed. In FIG. 1 the employment of a mixture of implants 36 of different sizes is shown.

Preferably implants 36 are comprised of a discrete, biodurable elastomeric matrix which is at least partially reticulated with inter-connected open-pored elements of defined shape and of known dimension so that a suitable number to fill a target site may be pre-selected according to the available information about the volume and shape of the target site. Each implant 36 also usefully comprise a resiliently compressible elastomeric matrix that regain at least substantially its shape after delivery to a biological site such that the implant 36, when compressed from a relaxed configuration to a first, compact configuration for delivery via a delivery device, expands to a second, working configuration in vitro.

Employment of a considerable number, for example, a group of from about 1 to about 200, or even about 30 or more, fluid-pervious elastomeric reticulated implants that are relatively small compared with the target site can be advantageous in facilitating desirable filling of the anisotropic sac geometry of a typical AAA or other problematic vascular site. This is necessitated by the extreme difficulty in delivering a single or a few large implants through a long narrow and/or small diameter catheter, needle, or cannula. The endoleak treatment sites are at times made more difficult to access due to the narrow passage and lack of maneuverability in the space surrounding the pre-existing endograft or the endograft that is put in prior to the implants being inserted for prophylactic or perioperative treatments for endovascular problems. Also, it will be easier to fill or substantially fill the aneurysm sac with smaller implants given the anisotropic irregular size and shape of the aneurysm sac. By use of such a group of small, low density, compressible implants, good accommodation of the implanted matrix to the geometry of an anisotropic or other target site may be obtained.

The structure of implants 36 comprises a reticulated inter-connected morphology can support cell growth and permit cellular ingrowth and proliferation in vivo and are useful as in vivo biological implantable devices, for example, for treatment of vasculature problems that may be used in vitro or in vivo to provide a substrate for cellular propagation. Optionally, the implants may be thrombogenic. It is also preferable that the implant matrix material have a microstructure intended to promote cellular proliferation and tissue ingrowth into, and preferably throughout the interior of the implant. In one embodiment, the reticulated elastomeric matrix of the invention facilitates tissue ingrowth by providing a surface for cellular attachment, migration, proliferation and/or coating (e.g., collagen) deposition. In another embodiment, any type of tissue can grow into an implantable device comprising a reticulated elastomeric matrix of the invention, including, by way of example, epithelial tissue, connective tissue, fibrovascular tissue or any combination thereof. In another embodiment of the invention, an implantable device comprising a reticulated elastomeric matrix of the invention can have tissue ingrowth substantially throughout the volume of its interconnected pores. Over time, this induced fibrovascular entity resulting from tissue ingrowth can cause the implantable device to be incorporated into the conduit. It may also prevent recanalization of the conduit.

Biodurable elastomeric reticulated implants 36 can be deployed throughout the aneurysm volume 38, around endograft 28 in all directions that are permitted by the local anatomy, may follow the aneurysm topography and may occupy pockets or occlusions such as crutch volume 40 beneath the bifurcation in aorta 10. Use of small implants 36 in such a manner can enable the occupation, by one or more implants 36, or by a portion of an implant, of pockets, folds or occlusions in the aneurysm volume that may have been undetected during imaging or have developed subsequently. In one embodiment, both smaller and larger implants may be, compacted and sufficiently held in place, by previously delivered neighboring implants and/or the local anatomy.

When constructed and deployed in accordance with the principles of the invention, biodurable elastomeric reticulated implants 36 can fill or substantially fill aneurysm volume 38 or the target site or space and slow or resist the flow or other movement of blood within the target 38. In one embodiment, aneurysm volume 38 is filled or packed to an extent that no implant additional to those already delivered can be received into aneurysm volume 38 wherein, preferably, the wall 26 of the aneurysm volume 38 is supported at multiple locations by contact with implants 36 so as to dampen or restrict movement of the wall. In another embodiment, aneurysm volume 38 or the target site or space is over-filled or over-packed with implants 36. In another embodiment, aneurysm volume 38 or the target site or space is under-filled or under-packed with implants 36. One useful degree of fill is such that none of the implants has freedom of movement in the target site, each being restrained from moving by its neighbor or the local anatomy. However initially, at least the first-arriving implant and probably up to fifty percent or more of the number of implants in the group selected to treat the target site is free to find its own orientation. Once the site is partially or completely filled, depending upon the size and number of implants 36, there may be a significant number that do not contact endograft 28.

While some benefit may be obtained by partially filling the aneurysm site, complete filling or substantially complete filing or partial overfilling or substantial overfilling is preferred. Also useful is substantial filling of the aneurysm wherein the implants effectively brace the aneurysm wall 26 in a number of locations spaced around the site and damping or otherwise controlling pulsatile movement of the aneurysm wall, yet have limited freedom to adjust their orientations or otherwise move relative to one another. Such substantial fill or loose packing may provide one or more bridges of implant material extending between the endograft and the aneurysm or other target vessel wall to brace the wall. Without being bound by any particular theory, the inventive method is practiced so that the cumulative effects of a group of implants 36 on blood movement in the target 38 reduce pressure on the aneurysm wall 26 or reduce hemodynamic perturbations in the target 38 that may stress aneurysm wall 26 and cause distention thereof or other undesirable effects.

Method embodiments of the invention include introducing a plurality of shaped reticulated elastomeric implants 36 into the perigraft space to substantially fill the aneurysm. Thus, in one desirable embodiment of the inventive method, implants are continually introduced into the target volume until it is no longer reasonably possible to insert them. In some cases, over-packing also may be allowed or necessary. In other cases, substantial over-packing also may be allowed or necessary. In another embodiment, the filling or packing of the targeted vascular site and the degree of packing are monitored by angiogram or angiography and is continued until angiographic outcome of “no flow” is achieved. In one embodiment, one or more remote or inaccessible pockets or corners of the aneurysm may not be occupied or may not be fully occupied by the implants. Furthermore, it is contemplated that there may be some lost space between adjacent implants, even when contacting one another. The degree to which the aneurysm is filled can be such as may be achieved without undue difficulty and without risk of collateral damage or rupturing of the target vessels or accessibility in the target space.

Embodiments of the invention include delivering reticulated elastomeric implants to a target site and releasing the implants into the target site with the location and orientation of each individual implant being determined by the local anatomy, by an endograft, if employed, and by neighboring implants. Thus, the location and orientation of a particular implant, or any implant, may not be predetermined, but may be passively determined by the implant according to the environment into which it is introduced. In general, but without excluding the possibility, the implants employed in the invention do not need to be actively secured or attached to any ambient structure at the target site. However, it is contemplated that some embodiments of the invention will sufficiently fill or pack the target site with implants that most, if not all, the implants will be held in position by their neighbors, the site anatomy, or an endograft or other prosthetic. Advantageously the implants can be formed of a biodurable material to promote permanent sac occlusion and endoleak resolution.

In FIG. 5, similar anatomy and structures bear the same reference numerals as are employed in FIG. 1 and that structure need not be described again. In this embodiment, aortic aneurysm 20 extends along the patient's lefthand common iliac artery 12 to the meeting point with internal iliac artery 16 and endograft 28 bypasses lefthand internal iliac artery 16 cutting it off from the aortic flow. However, if not controlled, lefthand internal iliac artery 16 can enable blood to backflow into aneurysm 20. Perhaps as many as 30 percent of patients with AAAs exhibit development of the aneurysm along a common iliac artery.

Also shown in FIG. 5 are several feeder arteries 56 that open into the upper aorta 10 and may include the lumbar, and inferior mesenteric arteries. Feeder arteries 56 can also be sources of Type II endoleakage, providing backflow into aneurysm volume 38.

In FIG. 5, implants 36 are shown generally by the shading within the aneurysm volume 38 which shading can be understood to indicate a group of implants 36, selected to treat volume 38, in the manner described in relation to FIG. 1. By employing the devices and apparatus of the invention to fill or substantially fill aneurysm volume 38 with reticulated elastomeric implants 36, the entry point of a feeder artery such as one of feeder arteries 56 can be occluded by the reticulated material of one or more implants 36. Such an occluding implant 36 may initially be beneficial in slowing blood flow from the feeder artery. In time, tissue ingrowth into the implant, fostered or accommodated by the implant material and structure may lead to complete occlusion of the feeder and blockage of flow from. Tissue growth stimulated as an element of the natural foreign body reaction of the host to the presence of implants 36 may also occur between individual implants 36 or between one or more implants 36 and the host anatomy, contributing to such blockage.

It is also contemplated that the described endoleak treatment method of the invention can be effective to seal an endoleak or endoleaks at the target site by occluding the inflow and outflow of blood through feeding and draining vessels. While the invention is not bound by any particular theory, nor limited to such an embodiment, it is contemplated that substantially filling the target site with biodurable elastomeric reticulated implants 36 in a substantial state of compression can be particularly effective in sealing endoleaks and occluding feeding and draining vessels.

However, if desired, occlusion of side branch or feeder and/or drainer vessels at the target site can also be effected by delivering one or more relatively large implants of biodurable elastomeric reticulated material to the target site and configured to extend over a significant area of, and conform with, a substantial portion of the internal peripheral surface of the target site. Use of single or multiple implants can be additionally effective in occluding small vessels of the vasculature that may open or drain into aneurysm walls 26. These small vessels may be sources of endoleaks. Suitably constructed, delivered and positioned, such a side branch occluding implant can occlude one or more side vessels opening into the respective peripheral area which may be a source of endoleaks. Such side branch occluding implants can be relatively thin and sheet-like, or laminar or cap- or bowl-like in shape and may cooperate with one or more other implants in the target site. Alternatively, the side branch occluding implants having a surface oriented in situ to conform with the target site internal surface may have a significant third dimension to help fill the target site.

In another aspect the invention solves these problems by providing a device or method for the treatment or prevention of endoleaks leading into or draining into a target vascular site such as an aneurysm, the device or method comprising delivering a single or plurality of reticulated, fluid-pervious elastomeric implants in a compressed state, into the target site and which recovery partially or substantially on release from the delivery system.

In another embodiment of late, post-operative endoleak treatment method such as occlusion or embolization of side branch or feeder and/or drainer vessels at the target site according to the invention, wherein the patient's condition comprises discrete, localized endoleaks that can be precisely located and accessed, a relatively small number of biodurable reticulated elastomeric implants, for example, from one to about ten implants, preferably from 1 to about 4 implants, are delivered to a target site within the sac to embolize the nexus of the endoleak or endoleaks. Highly compressible implants can be employed in such numbers.

Suitable matrices for such side branch occluding implants include biodurable elastomeric reticulated with inter-connected open-pored elements of defined shape and of known dimension. The suitable materials are resiliently compressible that allow for it to regain its shape after delivery to a biological site such that the implant 36, when compressed from a relaxed configuration to a first, compact configuration for delivery via a delivery device, expands to a second, working configuration. Preferred, however, are matrices have substantially similar materials characteristics to those of implant 36 and comprise of a reticulated inter-connected morphology can support cell growth and permit cellular ingrowth and proliferation in vivo. Alternately, they permit tissue ingrowth, either superficially or into the interior mass of the implant, as described herein, or as known to those skilled in the art. Such implants can be used to supplement known endograft implantation procedures that are found to be not fully effective with regard to endoleaks, if desired.

Sizing of the occlusion of side branch or filling aneurysm sac implants with respective target vessel space can be influenced by many factors, such as swelling of the device and/or natural extension of the ducts and arteries or relaxation of the surrounding endovascular and peripheral tissues in addition to or over the volume of the targeted vascular site. While not bound by any particular theory, it is possible that the implant may inherently swell up to 3% or in another embodiment up to 10%. It is also possible that the ducts and arteries, endovascular or peripheral wall tissues can naturally extend or swell or relax up to 5% in one embodiment, or up to 15% in another embodiment, or up to 30% in a further embodiment and up to 60% in another embodiment.

In most embodiments of the invention relating to filling or substantially filling of the aneurysm sac volume or the target site or space the in situ with multiple implants such as 2 or more implants per target site, volume of each individual implant is substantially less than the target volume, for example, less than at least about 25% percent of the target volume, preferably less than at least about 50% percent of the target volume and more preferably less than 90 percent of the target volume.

It is contemplated, in another embodiment, that even when their pores become filled with biological fluids, bodily fluids and/or tissue in the course of time, such implantable devices for vascular malformation applications and the like do not entirely fill the biological site in which they reside and that an individual implanted elastomeric matrix 36 will, in many cases, although not necessarily, have a volume of no more than 50% of the biological site within the entrance thereto. In another embodiment, an individual implanted elastomeric matrix 36 will have a volume of no more than 75% of the biological site within the entrance thereto. In another embodiment, an individual implanted elastomeric matrix 36 will have a volume of no more than 95% of the biological site within the entrance thereto.

Employing smaller or larger implants, the numbers can be adjusted accordingly. In one embodiment, the implants may not be selected to completely fill and obliterate the aneurysm sac or other target volume, but the total volume of the implants prior to compression and delivery may be selected to occupy a proportion of the target volume, for example, from about 20 to about 60 percent of the target volume. In another embodiment, the total volume of the implants prior to compression and delivery may be selected to occupy from about 60 to about 90 percent of the target volume. In another embodiment, the implants may be selected to occupy from about 90 to about 110 percent of the target volume. In another embodiment, the implants may be selected to occupy from about 90 to about 99 percent of the target volume. In another embodiment, the total volume of the implants prior to compression and delivery may be selected to occupy from about 99 to about 110 percent of the target volume. In another embodiment, the total volume of the implants prior to compression and delivery may be selected to occupy from about 110 to about 150 percent of the target volume. In another embodiment, the total volume of the implants prior to compression and delivery may be selected to occupy from about 150 to about 200 percent of the volume. It will be understood, however, that the invention also contemplates embodiments wherein such relatively small numbers of implants are adequate to fill or possibly obliterate the target site.

Though not bound by any particular theory, it can be expected that the target vessel or vascular condition may expand if necessary to accommodate the implants in case the total volume of the implants prior to compression and delivery and/or after recovery is larger than the target vessel or vascular condition or vascular malformation. In one embodiment, after the implants have been delivered to the target site and have expanded from their compressed state during delivery and when their pores become filled with biological fluids, bodily fluids and/or tissue in the course of time, such implants for vascular malformation applications have a volume of more than about 60% of the biological site in which they reside or within the entrance thereto. In another embodiment, after the implants have been delivered to the target site and have expanded from their compressed state during delivery and when their pores become filled with biological fluids, bodily fluids and/or tissue in the course of time, such implants for vascular malformation applications have a volume of more than about 80% of the biological site in which they reside or within the entrance thereto. In another embodiment, after the implants have been delivered to the target site and have expanded from their compressed state during delivery and when their pores become filled with biological fluids, bodily fluids and/or tissue in the course of time, such implants for vascular malformation applications have a volume of more than about 95% of the biological site in which they reside or within the entrance thereto. In another embodiment, after the implants have been delivered to the target site and have expanded from their compressed state during delivery and when their pores become filled with biological fluids, bodily fluids, and/or tissue in the course of time, such implants for vascular malformation applications have a volume of more than about 98% of the biological site in which they reside or within the entrance thereto. In another embodiment, after the implants have been delivered to the target site and have expanded from their compressed state during delivery and when their pores become filled with biological fluids, bodily fluids and/or tissue in the course of time, such implants for vascular malformation applications have a volume of more than about 105% of the biological site in which they reside or within the entrance thereto. In another embodiment, after the implants have been delivered to the target site and have expanded from their compressed state during delivery and when their pores become filled with biological fluids, bodily fluids and/or tissue in the course of time, such implants for vascular malformation applications have a volume of more than about 125% of the biological site in which they reside or within the entrance thereto. In another embodiment, after the implants have been delivered to the target site and have expanded from their compressed state during delivery and when their pores become filled with biological fluids, bodily fluids and/or tissue in the course of time, such implants for vascular malformation applications have a volume of more than about 135% of the biological site in which they reside or within the entrance thereto. In yet another embodiment, after the implants have been delivered to the target site and have expanded from their compressed state during delivery and when their pores become filled with biological fluids, bodily fluids and/or tissue in the course of time, such implants for vascular malformation applications have a volume of more than about 150% of the biological site in which they reside or within the entrance thereto. In yet another embodiment, after the implants have been delivered to the target site and have expanded from their compressed state during delivery and when their pores become filled with biological fluids, bodily fluids and/or tissue in the course of time, such implants for vascular malformation applications have a volume of more than about 200% of the biological site in which they reside or within the entrance thereto.

Furthermore, the invention includes treatment methods wherein the available volume of the target is substantially packed with compressed resilient implants delivered from a suitable introducer instrument.

According to the invention endovascular treatment devices and methods utilizing arterially deliverable implants are provided that are resistant to recanalization and migration. Arterial delivery via a catheter, or other introducer, is a relatively low-trauma procedure that can be employed post-operatively to address complications of more invasive measures such as the surgical implantation of vascular grafts and also, in the case of catheter-delivered endovascular grafts that are minimally invasive, is an alternative to open surgical repair. It will be understood that in most cases, implants designed for arterial delivery can, if desired, be delivered percutaneously, for example, as an adjunct to a more substantial surgical procedure.

In other embodiments such as those relating to occlusion of side branch or feeder or drainer vessels, with lesser number of implants such as 1 to 4 implants per target site, the total volume of the implants prior to compression and delivery and/or after recovery is more than about 85% percent of the target volume of the vascular site, preferably more than about 98% percent of the target volume of the vascular site, more desirably more than about 102% percent of the target volume of the vascular site, and most preferably more than about 125% percent of the target volume of the vascular site. In another embodiment relating to occlusion of side branch or feeder or drainer vessels, with a lesser number of implants such as 1 to 4 implants per target site, the total volume of the implants prior to compression and delivery and/or after recovery is more than about 135% percent of the target volume of the vascular site.

In yet another embodiment, in those cases, relating to occlusion of side branch or feeder or drainer vessels, with the number of implants of ranging from 1 to 4 implants per target site, the total volume of the implants prior to compression and delivery and/or after recovery is more than about 150% percent of the target volume of the vascular site. In yet another embodiment, in those cases, relating to occlusion of side branch or feeder or drainer vessels, with number of implants of ranging from 1 to 4 implants per target site, the total volume of the implants prior to compression and delivery and/or after recovery is more than about 200% percent of the target volume of the vascular site.

Implants 36 are delivered to aneurysm volume 38 or vascular occlusion site in a compressed state and expand at the site to partially or wholly regain their initial, uncompressed volume, or their relaxed volume adjusted for compression set. Some or all of implants 36 may remain impacted, or compacted, in situ, which is to say they do not fully recover their volumes prior to compression. In one embodiment, elastomeric matrices of the invention have sufficient resilience to allow substantial recovery, e.g., to at least about 50% of the size of the relaxed configuration in at least one dimension, after being compressed for implantation in target vascular defect such as aneurysm or endoleaks, and in certain cases sufficient strength and flow-through for the matrix to be used for controlled release of pharmaceutically-active agents, such as a drug, and for other medical applications. In another embodiment, elastomeric matrices of the invention have sufficient resilience to allow recovery to at least about 60% of the size of the relaxed configuration in at least one dimension after being compressed for implantation in the human body. In another embodiment, elastomeric matrices of the invention have sufficient resilience to allow recovery to at least about 90% of the size of the relaxed configuration in at least one dimension after being compressed for implantation in target vessels. In another embodiment, elastomeric matrices of the invention have sufficient resilience to allow recovery to at least about 97% of the size of the relaxed configuration in at least one dimension after being compressed for implantation in target vessels.

Implants 36 are elastomeric and can be delivered to aneurysm volume 38 or to a vascular occlusion site in a compressed state and can be compressed to at least about 97% of the size of the relaxed configuration volume. In another embodiment, implants 36 are elastomeric and can be delivered to aneurysm volume 38 or to a vascular occlusion site in a compressed state and can be compressed to at least about 95% of the size of the relaxed configuration volume. Implants 36 are elastomeric and can be delivered to aneurysm volume 38 or to a vascular occlusion site in a compressed state and can be compressed to at least about 90% of the size of the relaxed configuration volume. Implants 36 are elastomeric and can be delivered to aneurysm volume 38 or to a vascular occlusion site in a compressed state and can be compressed to at least about 80% of the size of the relaxed configuration volume. Implants 36 are elastomeric and can be delivered to aneurysm volume 38 or to a vascular occlusion site in a compressed state and can be compressed to at least about 70% of the size of the relaxed configuration volume. Implants 36 are elastomeric and can be delivered to aneurysm volume 38 or to a vascular occlusion site in a compressed state and can be compressed to at least about 50% of the size of the relaxed configuration volume.

Implants 36 are elastomeric and can be delivered to aneurysm volume 38 or to a vascular occlusion site in a compressed state and can be compressed to at least about 97% of the size of the relaxed configuration in at least one dimension. Implants 36 are eleasomeric and can be delivered to aneurysm volume 38 or to a vascular occlusion site in a compressed state and can be compressed to at least about 95% of the size of the relaxed configuration in at least one dimension. Implants 36 are elastomeric and can be delivered to aneurysm volume 38 or to a vascular occlusion site in a compressed state and can be compressed to at least about 90% of the size of the relaxed configuration in at least one dimension. Implants 36 are elastomeric and can be delivered to aneurysm volume 38 or to a vascular occlusion site in a compressed state and can be compressed to at least about 80% of the size of the relaxed configuration in at least one dimension. Implants 36 are elastomeric and can be delivered to aneurysm volume 38 or to a vascular occlusion site in a compressed state and can be compressed to at least about 70% of the size of the relaxed configuration in at least one dimension. Implants 36 are elastomeric and can be delivered to aneurysm volume 38 or to a vascular occlusion site in a compressed state and can be compressed to at least about 50% of the size of the relaxed configuration in at least one dimension.

Implants 36 are elastomeric and can be delivered to aneurysm volume 38 or to a vascular occlusion site in a compressed state and can be compressed to at least about 80% of the size of the relaxed configuration in at least two dimensions. Implants 36 are eleasomeric and can be delivered to aneurysm volume 38 or to a vascular occlusion site in a compressed state and can be compressed to at least about 75% of the size of the relaxed configuration in at least two dimensions. Implants 36 are elastomeric and can be delivered to aneurysm volume 38 or to a vascular occlusion site in a compressed state and can be compressed to at least about 70% of the size of the relaxed configuration in at least two dimensions. Implants 36 are elastomeric and can be delivered to aneurysm volume 38 or to a vascular occlusion site in a compressed state and can be compressed to at least about 60% of the size of the relaxed configuration in at least two dimensions. Implants 36 are elastomeric and can be delivered to aneurysm volume 38 or to a vascular occlusion site in a compressed state and can be compressed to at least about 50% of the size of the relaxed configuration in at least two dimensions.

In one embodiment, the biodurable reticulated elastomeric implant can recover in a resilient fashion and can expand from the first, compact configuration to the second, working configuration over a short time, e.g., about 95% recovery in 90 seconds or less in one embodiment, or in 40 seconds or less in another embodiment, or in 20 seconds or less in yet another embodiment, each from 75% compression strain held for up to 10 minutes. In another embodiment, the expansion from the first, compact configuration to the second, working configuration occurs over a short time, e.g., about 95% recovery in 180 seconds or less in one embodiment, in 90 seconds or less in another embodiment, in 60 seconds or less in another embodiment, each from 75% compression strain held for up to 30 minutes. In another embodiment, the biodurable reticulated elastomeric implant recovers in about 10 minutes to occupy at least 97% of the volume occupied by its relaxed configuration, following 75% compression strain held for up to 30 minutes.

In one embodiment all of the biodurable elastomeric reticulated implants for packing the aneurysm sac, embolizing the endoleak nexus within the sac and occluding the feeder vessels such as collateral arteries that drain into the aneurysm sac can be delivered via catheter, cannula, endoscope, arthoscope, laproscope, cystoscope, syringe or other suitable delivery-device and can be satisfactorily implanted or otherwise exposed to living tissue and fluids for extended periods of time, for example, at least 29 days, preferably for at least several weeks and most preferably at least two to five years or more.

The inventive implantable device is reticulated, i.e., comprises an interconnected network of pores and/or voids, by being formed having a reticulated structure and/or by undergoing a reticulation process. In another embodiment, a material may be described as reticulated, comprising a continuous network of solid structures, such as struts and intersections without any significant terminations, isolated zones or discontinuities, other than at the boundaries of the elastomeric matrix, in which network a hypothetical line may be traced entirely through the material of solid phase from one point in the network to any other point in the network. In another embodiment, a void phase formed or at least partially bounded by the struts and intersections is also a continuous network of interstitial spaces, or intercommunicating fluid passageways for gases or liquids, which fluid passageways extend throughout and are defined by (or define) the structure of solid phase of elastomeric biodurable reticulated matrix or the implants and open into all its exterior surfaces. In other embodiments, as described above, there are only a few, substantially no, or no occlusions or closed cell pores that do not communicate with at least one other pore in the void network.

In one embodiment, reticulation of a product of the invention, if not already a part of the described production process for making the implants or the materials from which the implants are made or fabricated, may be used to remove at least a portion of any exiting interior “windows”, i.e., the residual cell walls. Foam materials with some ruptured cell walls are generally known as “open-cell” materials or foams. In contrast, materials known as “reticulated” or “at least partially reticulated” have many, i.e., at least about 40%, of the cell walls that would be present in an identical porous material except composed exclusively of cells that are closed, at least partially removed. Where the cell walls are least partially removed by reticulation, adjacent reticulated cells open into, interconnect with, and communicate with each other. Materials from which more, i.e., at least about 65%, of the cell walls have been removed are known as “further reticulated”. If most, i.e., at least about 80%, or substantially all, i.e., at least about 90%, of the cell walls have been removed then the material that remains is known as “substantially reticulated” or “fully reticulated”, respectfully. It will be understood, that, pursuant to this art usage, a reticulated material or foam comprises a network of at least partially open interconnected cells.

Reticulation provides fluid permeability throughout the implantable device and permits cellular ingrowth and proliferation into the interior of the implantable device. Reticulation tends to increase porosity and fluid permeability. In one embodiment the microstructure of biodurable elastomeric reticulated implant is constructed to permit or encourage cellular adhesion to the surfaces of solid phase such as struts and intersections, neointima formation thereon and cellular and tissue ingrowth and proliferation into pores and/or voids, when biodurable elastomeric reticulated implant or the material matrix resides in suitable in vivo locations for a period of time.

Without being bound by any particular theory, it is thought when the reticulated elastomeric implants are placed in or carried to a conduit or a vessel through body fluid passes or accumulates such as the targeted aneurysm sac or side branch or feeder and/or drainer vessels, it will provide an immediate resistance to the flow of body fluid such as blood. This will be associated with an inflammatory response and the activation of a coagulation cascade leading to formation of a clot, owing to a thrombotic response. Thus, local turbulence and stagnation points induced by the implantable device surface may lead to platelet activation, coagulation, thrombin formation and clotting of blood. The natural process of thrombosis will be induced due to the presence of the implant and will initiate the first step of dealing with endoleakage or sac therapy. Without being bound by any particular theory, it is believed that the thrombotic and/or inflammatory response will assist in initial migration resistance of the implant in the conduit such as a targeted aneurysm sac or side branch or feeder and/or drainer vessels.

In one embodiment, cellular entities such as fibroblasts and tissues can invade and grow into reticulated elastomeric implants such as those represented by implants 36. In due course, such ingrowth can extend into the interior pores and interstices of the inserted reticulated elastomeric implants. Eventually, elastomeric implant can become substantially filled with proliferating cellular ingrowth that provides a mass that can occupy the site or the void spaces in it. Over time, this induced fibrovascular entity resulting from tissue ingrowth can cause the implantable device to be incorporated into the conduit. In one embodiment, such implantable devices can also eventually become integrated, e.g., ingrown with tissue or will become bio-integrated. The types of tissue ingrowth possible include, but are not limited to, fibrous tissues and endothelial tissues.

Over time, this induced fibrovascular entity resulting from tissue ingrowth can cause the implantable device to be incorporated into the conduit. In another embodiment the reticulated morphology or micro-structure will allow for the implantable device to become completely ingrown and proliferated with cells and fibrous tissues and possibly seal off such features in a biologically sound, effective, and lasting manner. With such ingrown and proliferated tissue the implant will be able to integrate to the host tissue in the lumen and will have a very low possibility of migration, thereby not negating nor reversing the occlusion process. Without being bound by any particular theory, matrices or implants without inter-connected pores or reticulated mophology or reticulation, the implant will not be able to integrate to the host tissue in the lumen and will have a very high possibility of migration or a blow-out as the pressure builds up with the obstructed fluid thereby negating or reversing the occlusion process. Some implants might allow for tissue penetration for the first few surface layers but not beyond and would still lead to poor integration with to the host tissue in the lumen and will thus have a very high possibility of migration or a blow-out as the pressure builds up with the obstructed fluid thereby negating or reversing the occlusion process.

In another embodiment, tissue ingrowth and proliferation may also prevent recanalization of the conduit. In another embodiment, the tissue ingrowth is scar tissue which can be long-lasting, innocuous and/or mechanically stable. In another embodiment, over the course of time, for example, for 2 weeks to 3 months to 1 year, reticulated elastomeric implant may be completely filled and/or integrated with tissue, fibrous tissue, scar tissue, or the like. Tissue ingrowth can lead to incorporation and integration with the body lumen or surrounding vessels or tissues and very effective resistance to migration of the implantable device and re-canalization over time.

The presence of implants 36 in aneurysm volume 38 desirably may result in initiation of a foreign body host reaction, with minimal, or only modest, inflammatory response, permitting tissue ingrowth into the interiors of the implants 36. Pursuant to the invention herein and the inventions of the related applications, implants 36, desirably, are fabricated of a suitable material, are constructed, and optionally may be treated, to permit or promote such tissue ingrowth not only into marginal volumes of implants 36, but also into the interiors of the implants. Suitable structural characteristics facilitating such ingrowth are further described hereinbelow. Extensive and effective tissue ingrowth can fix the implants in position in aneurysm 20, as is also described in more detail hereinbelow. These eventualities can result in effective occlusion of the target vascular site and even, its obliteration. In time, target vessel site such as endoleak, aneurysm sac 20 may, in some cases, be converted to a solid mass of “healthy” scar tissue with risks of serious adverse aneurysm-related events being substantively reduced or even eliminated.

In one embodiment all of the elastomeric reticulated implants for packing the aneurysm sac, embolizing the endoloeak nexus within the sac and occluding the feeder vessels such as collateral arteries that drain into the aneurysm sac are biodurable or constructed from materials are also biodurable. Useful elastomers and other matrix materials or products that are biostable for extended periods of time in a biological environment, are described herein as “biodurable” in the present application, Particularly useful embodiments of such materials for employment in the practice of the present invention do not exhibit significant symptoms of breakdown or degradation, erosion or deterioration of useful mechanical properties relevant to their employment when exposed to biological environments for desired periods of time. The periods of implantation may be, for example, for 29 days or more. The periods of implantation on the other hand may be, for example, several weeks, months, for example, at least six months, or years, for example, at least two years, five years or more, the lifetime of a host product in which the elastomeric products of the invention are incorporated such as a graft or prosthetic, or the lifetime of an animal host to the elastomeric product.

However, some amount of cracking, fissuring or a loss in toughness and stiffening for the implants—at times referred to as ESC or environmental stress cracking—may not be relevant to endovascular and other uses as described herein. Many in vivo applications, e.g., when used as implant 36 for treatment of vascular abnormalities, expose it to little, if any, mechanical stress and, thus, are unlikely to result in mechanical failure leading to serious patient consequences. Accordingly, the absence of ESC may not be a prerequisite for biodurability of suitable elastomers in such applications for which the present invention is intended because elastomeric properties become less important as endothelialization and cellular ingrowth and proliferation advance.

In one embodiment all of the elastomeric reticulated implants for packing the aneurysm sac, embolizing the endoloeak nexus within the sac and occluding the feeder vessels such as collateral arteries that drain into the aneurysm sac are biocompatible or constructed from materials are also biocompatible in the sense of inducing few, if any, adverse biological reactions when implanted in a host patient. To that end, in another embodiment for use in the invention, implants or the materials they are made from are free of biologically undesirable or hazardous substances or structures that can induce such adverse reactions or effects in vivo when lodged in an intended site of implantation for the intended period of implantation. Such implants or the materials they are made from accordingly should either entirely lack or should contain only very low, biologically tolerable quantities of cytotoxins, mutagens, carcinogens and/or teratogens. In another embodiment, biological characteristics for biodurability of elastomers to be used for fabrication of elastomeric reticulated implant include at least one of resistance to biological degradation, and absence of or extremely low: cytotoxicity, hemotoxicity, carcinogenicity, mutagenicity, or teratogenicity. Furthermore, it is desirable elastomeric implants retain such favorable biocompatibility without adverse immunological or other undesired reactions properties throughout their useful life.

It will be understood from the foregoing descriptions that biodurability and biocompatibility are different properties although certain chemical characteristics may be relevant to, or may confer, both biodurability and biocompatibility. Some preferred embodiments are both biodurable and biocompatible in the foregoing senses.

In one embodiment all of the implants for packing the aneurysm sac, packing the peri-graft space, embolizing the endoloeak nexus within the sac and occluding the feeder vessels such as collateral arteries that drain or bring additional fluid or blood into the sac can be of similar size and shape. In another embodiment, the implants can come in a variety of sizes and shapes. Desirably also, implants are selected to be, in their resident state or after they have substantially recovered following delivery in compressed state, too large to migrate out of aneurysm volume along a collateral vessel. Preferably, implants are delivered into the aneurysm volume with a size, being the size attained once the implant is fully detached from its delivery device, which is a sufficient size to prevent such migration via a collateral vessel. In another embodiment, implants are selected to be, in their resident state or delivered into the aneurysm volume with a size, being the size attained once the implant is fully detached from its delivery device (and following delivery in compressed state), is too large to substantially or fully migrate out of the neck of the aneurysm or the openings in the aneurysm wall that connect to the aneurysm to the lumens or vessels carrying blood. The occupying body of implants can be selected to have sizes, shapes and configurations permitting catheter delivery and such as to occupy a significant or substantial proportion of the treatment volume or over-packing the treatment volume but, in most cases, not all, of the treatment volume, and to limit flow of blood in or through the treatment volume.

Individual ones of the shaped implants can have any one of a range of configurations, including cylindrical, cylindrical with hollow center, cylindrical with an annulus, conical, frustoconical, single tapered cylindrical, double tapered cylindrical being a cylindrical shape tapered at both ends, bullet-shaped, ring-shaped, C-shaped, S-shaped spiral, helical, spherical, spherical with hollow center, spherical with hollow not at the center, spherical with slits cut into them, elliptical, ellipsoidal, polygonal, star-like, compounds or combinations of two or more of the foregoing other such configuration as may be suitable, as will be apparent to those skilled in the art and solid and hollow embodiments of the foregoing. Other shapes include but not necessarily limited to rods, spheres, cubes, pyramids, tetrahedrons, cones, cylinders, trapezoids, parallelepipeds, ellipsoids, fusiforms, tubes or sleeves or a folded, coiled, helical or other more compact configuration. Hollow embodiments are contemplated as being useful as employing less porous material for given bulk volume of the implant, as defined by the outer peripheral surface of the implant than would a similarly sized “solid” implant, which is to say an implant whose whole volume is filled with porous material. In considering the bulk volume of an implant for the purposes of the invention, what is of interest is the volume the implant occupies in the target site and from which other implants are excluded, which bulk volume desirably may include interior hollow volumes, provided that the implant has a suitable configuration or conformation.

Preferred hollow embodiments can have an opening or an open face to permit direct fluid access to the interior of the bulk configuration of the implant. Other possible embodiments are set forth in co-pending, commonly assigned U.S. patent application Ser. No. 10/692,055, filed Oct. 22, 2003, which is incorporated herein by reference in its entirety. Still further possible embodiments of shaped implant include modifying the foregoing configurations by folding, coiling, tapering, or hollowing or the like to provide a more compact configuration when compressed, in relation to the volume to be occupied by the implant in situ. Implants having solid or hollowed-out, relatively simple elongated shapes such as cylindrical, bullet-like and tapered shapes are contemplated as being particularly useful in practicing the invention.

FIG. 2 represents a generally tubular implant 42 formed of a suitable reticulated elastomeric matrix material, as described elsewhere herein, having an outer periphery 44, or envelope, which is that of a right cylinder. The interior of implant 42 is sculpted out to enhance the overall compressibility of the implant 42, with an open-ended hollow volume 46, which can also be right cylindrical, or may have any other desired shape.

FIG. 3 illustrates a bullet-like implant 48 having an outer periphery 49 and a blind hollow volume 50. It is contemplated that a tapered or bullet-shaped outer profile, whether being solid or hollow, may facilitate catheter delivery. FIG. 4 illustrates a tapered, frusto-conical implant 52 which has an outer periphery 53 and an open-ended hollow volume 54. An optional annular wall 55 can be provided in the base of implant 52 to prevent nesting. Other than their shapes, implants 48 and 52 are generally similar to implant 42, and all three implants 42, 48 and 52 may have any desired external or internal cross-sectional shapes including circular, square, rectangular, polygonal and so on. Additional possible shapes are described hereinbelow. Alternatively, implants 42, 48 and 52 may be “solid”, with any of the described exterior shapes, being constructed throughout of reticulated material and lacking a hollow interior on a macroscopic scale. Preferably any hollow interior is not closed but is macroscopically open to the ingress of fluids, i.e., fluids can directly access the macroscopic interior of the implant structure, e.g., hollows 46, 50 or 54, and can also migrate into the implant through its pore network.

While shown as largely smooth, the outer peripheries 44, 49, and 53 of implants 42, 48, and 52, respectively, or of other useful shapes of implant 36, can have more complex shapes for desired purposes, for example, corrugated to promote interengagement between implants in situ, promoting stabilization of the target site.

Preferably the volumes of hollows 46, 50 and 54 relative to the implant bulk volumes are selected to enhance compressibility while still permitting implants 42, 48, and 52 to resist blood flow. Thus, the hollow interior volumes of the implants can constitute any suitable proportion of the respective implant volume, for example, in the range of from about 10 to about 90 percent with other useful volumes being in the range of about 20 to about 50 percent.

Shaping and sizing can include custom shaping and sizing to match an implantable device to a specific treatment site in a specific patient, for example, as determined by imaging or other techniques known to those in the art. The shape may be a working configuration, such as any of the shapes and configurations described in the copending applications, or the shape may be for bulk stock. Stock items may subsequently be cut, trimmed, punched or otherwise shaped for end use. The sizing and shaping can be carried out, for example, by using a blade, punch, drill, or laser. In another embodiment, the sizing and shaping can be carried out by machining. In each of these embodiments, the processing temperature or temperatures of the cutting tools for shaping and sizing such as blade, punch, drill or machining fixtures can be at ambient temperature and in certain cases the shaping and sizing can be facilitated by coolant or lubricant that can be easily washed away in a later cleaning step if required. In another embodiment, the processing temperature or temperatures of the cutting tools for shaping and sizing can be greater than about 100° C. In another embodiment, the processing temperature(s) of the cutting tools for shaping and sizing can be greater than about 130° C. Finishing steps can include, in one embodiment, trimming of macrostructural surface protrusions, such as struts or the like, which can irritate biological tissues. In another embodiment, finishing steps can include heat annealing. Annealing can be carried out before or after final cutting and shaping.

In yet another embodiment, the sizing and shaping of the implant can be partially or fully carried out by cryocutting or cryomachining by such processes as, e.g., freezing a block of foam with iospentane or liquid nitrogen or other suitable medium and then machining the implant. This can allow for more precise cutting and smaller sized implants under 1 mm.

The dimensions of the shaped and sized implants made from biodurable reticulated elastomeric materials can vary depending on the particular vascular malformation treated and implants are preferably selected to permit loading into a suitable introducer in a compressed state followed by recovery after delivery at the target site. In one embodiment, the major dimension or the maximum dimension of a device prior to being compressed and delivered is from about 0.5 mm to about 100 mm. In another embodiment, the major dimension or the maximum dimension of a device prior to being compressed and delivered is from about 2 mm to about 10 mm. In another embodiment, the major dimension of a device prior to being compressed and delivered is from about 3 mm to about 8 mm. In another embodiment, the major dimension or the maximum dimension of a device prior to being compressed and delivered is from about 8 mm to about 30 mm. In another embodiment, the major dimension of a device prior to being compressed and delivered is from about 30 mm to about 100 mm. Biodurable reticulated elastomeric materials can exhibit compression set upon being compressed and transported through a delivery-device, e.g., a catheter, syringe or endoscope. In another embodiment, compression set and its standard deviation are taken into consideration when designing the pre-compression dimensions of the device.

In another embodiment, the minimum dimension of the implant may be as little as 0.5 mm and the maximum dimension as much as about 200 mm or even greater. The largest transverse dimension or the diameter of suitable implants can have any appropriate value, for example, in the range of from about 1 to about 200 mm. Some embodiments of implant useful in the practice of the invention for this purpose can have a transverse dimension or the diameter in the range of from about 3 to about 20 mm. Other embodiments can have transverse dimensions or the diameter in the range of from about 5 to about 15 mm. In another embodiment, the longitudinal dimension can be from about 10 to about 200 mm. Those skilled in the art will understand suitable dimensions that can be employed. Useful dimensions can be in the range of, for example, from about 2 to about 50 mm.

Thus, the invention provides aneurysm treatment methods wherein a group of implants 36 is delivered to aneurysm 20 in such a manner as to occlude any, and preferably all, accessible and identified feeder arteries 56. Such feeder occlusion is difficult to achieve with known custom fabrication of a single implant shaped to fit a target site. In contrast, some preferred embodiments of the invention can employ two or more, more preferably ten or even twenty or more implants in a group of implants intended to treat a single site. The invention also provides one or more introducers, loaded, or repeatedly loaded, if necessary, with sufficient implants to constitute a desired group of implants for treatment of a target site.

If desired, or if necessary, lefthand internal iliac artery 16 can be occluded by a reticulated elastomeric implant implant plug 58 lodged within the lumen of lefthand internal iliac artery 16. Implant implant plug 58 can be formed of a matrix having a material and structure intended to permit or encourage tissue ingrowth, similarly to that employed for implants 36. In addition, or alternatively, implant implant plug 58 can be selected to be oversized in its relaxed, uncompressed dimensions so that it is a compression fit into the lumen. In a method embodiment of the invention, implant plug 58 is loaded into a catheter or the like with substantial lateral compression, as described above, to have significantly reduced lateral dimensions with respect to its relaxed state. “Lateral” can be understood to reference dimensions lateral to the extent of a lumen such as a side branch feeder and lateral to the direction of flow of fluid in the lumen. Accordingly, the method may, for example, comprise compressing a cylindrical implant plug 58 to a reduced diameter, optionally a diameter that can be accommodated in a catheter 60 or 62 capable of entering at least the mouth of the side branch vessel and the loading of the compressed implant plug into a distal cavity in the catheter. It will be understood that compression may be effected during or after loading into the catheter, if desired.

A suitable migration-resistant implant plug 58 can be implanted by deploying catheter 60 ipsilaterally via the patient's lefthand external iliac artery 14, along path 64, or by deploying catheter 62 contralaterally via the patient's righthand external iliac artery 14, along path 66. Catheter deployment may be effected by insertion of the catheter 60 or 62 loaded with compressed implant plug 58, into the patient's vasculature at a suitable point and manipulating catheter 60 or 62 to move its distal end 68 or 70 along path 64 or 66 receptively until the distal end 68 or 70 of catheter 60 or 62 enters or addresses mouth 72 of internal iliac artery 16, or another targeted side branch vessel. When distal end 68 or 70 is suitably located at mouth 72 or further along artery 16, plug-loaded catheter 60 or 62 is operated to discharge implant plug 58 from catheter 60 or 62 into internal iliac artery 16, for example, by manipulation of a plunger and optional actuation of a implant plug release mechanism, which may be effected simultaneously by said plunger manipulation, to push implant plug 58 out of catheter 60 or 62.

As it is discharged, implant plug 58 undergoes resilient recovery and expands or attempts to recover its precompression configuration, resulting in prestressed engagement of the outer implant plug surface or surfaces with the endothelial surfaces of internal iliac artery 16. Desirably, the degree of compression, liquid-permeability, outer surface frictional features and other relevant characteristics of implant plug 58 are selected with a view to ensuring that implant plug 58 remains lodged in position in artery 16. Once delivered and lodged in position located within iliac artery 16, implant plug 58 can initially slow the flow of blood in artery 16 and eventually become ingrown with tissue, providing a substantial or complete barrier to blood flow in the vessel.

Pursuant to the invention, implantation of implant plug 58 into internal iliac artery 16, or into another branch artery such as one or more of side branch arteries 56, or into another bodily lumen, to occlude the artery or other lumen can be effected for any desired purpose, in conjunction with the use of an endograft 28, or without the use of same, as desired. Novel methods and devices for occlusion of a bodily lumen with a compressed reticulated elastomeric plug, as described and suggested herein, provide another aspect of the invention which can be practiced independently of other aspects.

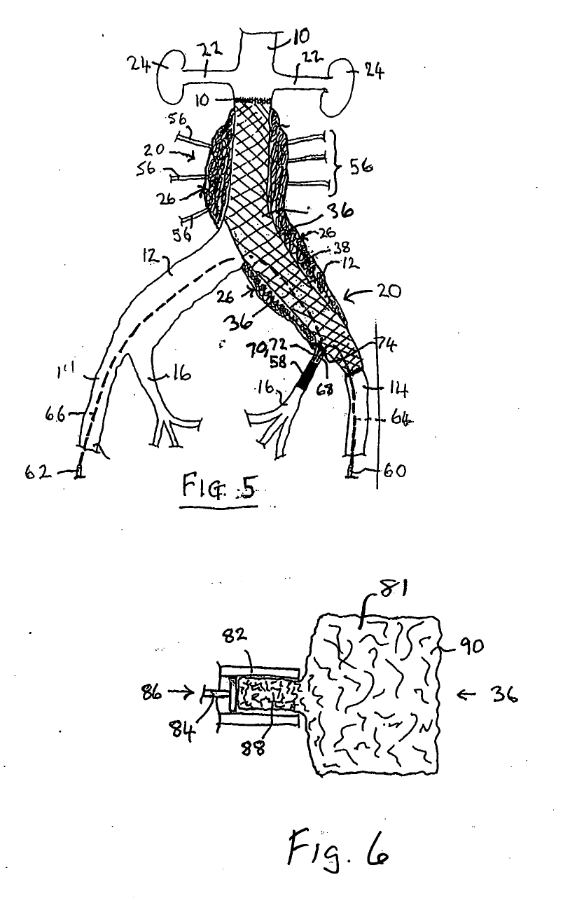

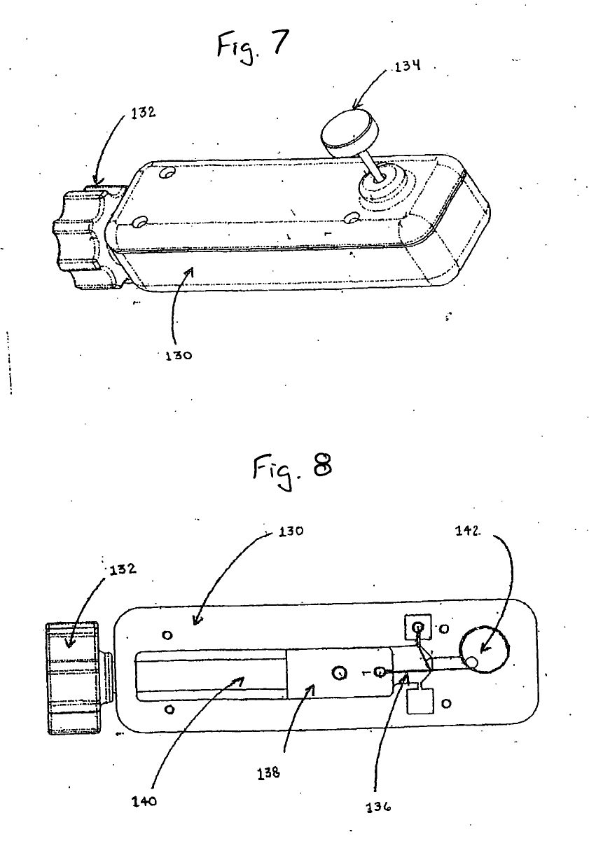

In FIG. 6 an implant 81 is partially discharged from a catheter 82 from which the implant 81 is being ejected by a plunger 84 moved, e.g., manually, in the direction of arrow 86. A compressed portion 88 of implant 81 remains within catheter 82, while that portion of implant 81 ejected from catheter 82 has promptly expanded as a result of its inherent resilience, becoming expanded portion 90. Further motion of plunger 84 in the direction of arrow 86 will discharge implant 81 completely from catheter 82, for example, into a target site such as aneurysm volume 38, with compressed portion 88 expanding as it emerges from catheter 82. A preferred embodiment is purposeful, slow deployment of the implant out of the catheter, for example, for a period of time ranging from about 3 seconds to about 2 minutes, preferably from about 10 to about 60 seconds, and more preferably from about 15 to 45 seconds. This will allow the implant to fully or substantially expand and will help to minimize the undesirable effects of distal embolization or migration of the implant, which may result while the implant is not yet fully recovered or expanded following rapid deployment out of the catheter. Substantial compression of implant 81 may result in a significant frictional force resisting discharge from catheter 82, depending upon the nature of the implant matrix and its length. Usefully, to mitigate the friction, catheter 82 can be highly polished and/or coated or formed of a low-friction material such as silicone or polytetraflueoroethylene.

For treatment of vascular malformations (such as aneurysm sac, endoloeak nexus within the sac and occluding the feeder vessels), it is an advantage of the invention that the implantable elastomeric matrix elements can be effectively employed without any need to closely conform to the configuration of the vascular malformation, which may often be complex and difficult to model. Thus, in one embodiment, the implantable elastomeric matrix elements of the invention have significantly different and simpler configurations.

The selection of suitable implants for inclusion in a group of implants to be delivered into a target cavity may be made on the basis of imaging, personal observation by the medical practitioner, or by other diagnostic methods such as CT scans. The selection may be determined or adjusted during an implant delivery procedure according to the number of implants 36 that can be accommodated or preferably to substantially pack or fill the target vascular site, such as aneurysm volume 38, or by other factors that become apparent or develop during the procedure. Thus, the surgeon or other practitioner may increase or decrease the number of implants to be delivered or use a different size of implant. In this, and other, ways the invention provides a flexible system for the treatment of vascular irregularities. The invention is not limited to a mechanical implementation of procedures devised in response to diagnostic conclusions based upon somatic conditions existing at a point in time prior to the moment of implant delivery but can permit the observations and judgments of the surgeon to be implemented in “real time.”

One broad aspect of the invention comprises a method for the treatment of late, or post-operative endoleaks that are identified after an endograft has been implanted. The existence of such late endoleaks can be identified in post-operative computerized tomography, “CT” scans that can be or are generally performed at regular intervals following an endograft procedure. Pursuant to the present invention, one method of treating late endoleaks comprises the introduction of an occupying body of individual, shaped implants into the aneurysm sac. The occupying body of implants can be selected to occupy a substantial proportion of the aneurysm sac in the perigraft space and to reduce blood flow or reduce the amplitude of hemodynamic forces acting on the aneurysm or other vascular wall.

These self-expandable conformal implants are machined from a block of biodurable elastomeric reticulated matrix using custom dies. The implants are preferably cylindrical in shape and may be tapered at one or both ends to allow the implants to be more easily loaded into the delivery catheters owing to ease of compressing the tapered ends to facilitate their entry or matching or mating with the delivery catheters, syringe, etc. Implants with flat non-tapered ends or slightly curved non-tapered ends can be somewhat difficult and challenging to compress and load into delivery catheters due to the difficulty in compressing larger cross-sections into small diameters or for entry or matching or mating with the small diameter delivery catheters, syringes, etc. In another embodiment, the VOD configuration, with no cuts, slots, or other irregularities, is designed to promote continuous contact with the vessel wall along the longitudinal length of the implant to minimize or prevent migration. Also, implants having cylindrical configurations at least partially, at times can facilitate machining.

Another embodiment of this invention, then, involves the use of a metallic frame to which a sufficient amount of reticulated elastomeric material is attached. The purpose of using a metallic frame to “house” the polymeric material is to minimize the amount of material required for occlusion, thereby offering a lower profile implant for compression into a suitable delivery catheter. It is also the purpose of the metallic frame to impart radiopacity to the implant. In this embodiment, instead of delivering an oversized polymeric implant which would be necessary to resist blood flow, a metallic frame enables the implant to be sized to the exact diameter and dimensions of the target vessel. The metallic frame may be in the form of a tubular structure similar to a stent, a helical or coil-like structure, an umbrella structure, or other structure generally known to those skilled in the art. The frame is preferably comprised of metals which have shape memory, including, but not limited to, nitinol. Attachment of the elastomeric material can be accomplished by means including, but not limited to, chemical bonding or adhesion, suturing, pressure fitting, compression fitting, and other physical methods.

Another aspect of this invention comprises enhanced implants that are reinforced with internal metallic support structures. These internal support structures are intended to ensure that the implant is properly placed and oriented within the vessel, that is, oriented longitundinally such that the central axis of the cylindrical implant is aligned in a parallel direction to the flow of the blood through the vessel. It is also the purpose of these internal metallic support structures to impart radiopacity to the implant. The internal support structure is embedded into the foam implant and may be in the form of a straight or curved wire, helical or coil-like structure, umbrella structure, or other structure generally known to those skilled in the art. The internal support structure is preferably comprised of metals with shape memory including, but not limited to, platinum and nitinol. Embedding of the support structure would be done subsequent to machining of the foam implant, and would be secured within the implant such that natural systolic forces experienced in the vasculature cannot dislodge or otherwise displace the structure.

Some materials suitable for fabrication of the implants according to the invention will now be described. Implants useful in this invention or a suitable hydrophobic scaffold comprise a reticulated polymeric matrix formed of a biodurable polymer that is elastomeric and resiliently-compressible so as to regain its shape after being subjected to severe compression during delivery to a biological site such as vascular malformations described here. The structure, morphology and properties of the elastomeric matrices of this invention can be engineered or tailored over a wide range of performance by varying the starting materials and/or the processing conditions for different functional or therapeutic uses.

The inventive implantable device is reticulated, i.e., comprises an interconnected network of pores and channels and voids that provides fluid permeability throughout the implantable device and permits cellular and tissue ingrowth and proliferation into the interior of the implantable device. The inventive implantable device is reticulated, i.e., comprises an interconnected and/or inter-communicating network of pores and channels and voids that provides fluid permeability throughout the implantable device and permits cellular and tissue ingrowth and proliferation into the interior of the implantable device. The inventive implantable device is reticulated, i.e., comprises an interconnected and/or inter-communicating network of pores and/or voids and/or channels that provides fluid permeability throughout the implantable device and permits cellular and tissue ingrowth and proliferation into the interior of the implantable device. The biodurable elastomeric matrix or material is considered to be reticulated because its microstructure or the interior structure comprises inter-connected and inter-communicating pores and/or voids bounded by configuration of the struts and intersections that constitute the solid structure. The continuous interconnected void phase is the principle feature of a reticulated structure.

Preferred scaffold materials for the implants have a reticulated structure with sufficient and required liquid permeability and thus selected to permit blood, or other appropriate bodily fluid, and cells and tissues to access interior surfaces of the implants. This happens due to the presence of inter-connected and inter-communicating, reticulated open pores and/or voids and/or channels that form fluid passageways or fluid permeability providing fluid access all through.

Preferred foams or at least partially hydrophobic reticulated, elastomeric polymeric matrix materials for fabricating implants according to the invention are flexible and resilient in recovery, so that the implants are also compressible materials enabling the implants to be compressed and, once the compressive force is released, to then recover to, or toward, substantially their original size and shape. For example, an implant can be compressed from a relaxed configuration or a size and shape to a compressed size and shape under ambient conditions, e.g., at 25° C. to fit into the introducer instrument for insertion into the vascular malformations (such as an aneurysm sac, endoloeak nexus within the sac and occluding the feeder vessels). Alternatively, an implant may be supplied to the medical practitioner performing the implantation operation, in a compressed configuration, for example, contained in a package, preferably a sterile package. The resiliency of the elastomeric matrix that is used to fabricate the implant causes it to recover to a working size and configuration in situ, at the implantation site, after being released from its compressed state within the introducer instrument. The working size and shape or configuration can be substantially similar to original size and shape after the in situ recovery.

Preferred scaffolds are reticulated elastomeric polymeric materials having sufficient structural integrity and durability to endure the intended biological environment, for the intended period of implantation. For structure and durability, at least partially hydrophobic polymeric scaffold materials are preferred although other materials may be employed if they meet the requirements described herein. Useful materials are preferably elastomeric in that they can be compressed and can resiliently recover to substantially the pre-compression state. Alternative reticulated polymeric materials with interconnected pores or networks of pores that permit biological fluids to have ready access throughout the interior of an implant may be employed, for example, woven or nonwoven fabrics or networked composites of microstructural elements of various forms.

A partially hydrophobic scaffold is preferably constructed of a material selected to be sufficiently biodurable, for the intended period of implantation that the implant will not lose its structural integrity during the implantation time in a biological environment. The biodurable elastomeric matrices forming the scaffold do not exhibit significant symptoms of breakdown, degradation, erosion or significant deterioration of mechanical properties relevant to their use when exposed to biological environments and/or bodily stresses for periods of time commensurate with the use of the implantable device. In one embodiment, the desired period of exposure is to be understood to be at least 29 days, preferably several weeks and most preferably 2 to 5 years or more. This measure is intended to avoid scaffold materials that may decompose or degrade into fragments, for example, fragments that could have undesirable effects such as causing an unwanted tissue response.

The void phase, preferably continuous and interconnected, of the reticulated polymeric matrix that is used to fabricate the implant of this invention may comprise as little as 50% by volume of the elastomeric matrix, referring to the volume provided by the interstitial spaces of elastomeric matrix before any optional interior pore surface coating or layering is applied. In one embodiment, the volume of void phase as just defined, is from about 70% to about 99% of the volume of elastomeric matrix. In another embodiment, the volume of void phase is from about 80% to about 98% of the volume of elastomeric matrix. In another embodiment, the volume of void phase is from about 90% to about 98% of the volume of elastomeric matrix.

As used herein, when a pore is spherical or substantially spherical, its largest transverse dimension is equivalent to the diameter of the pore. When a pore is non-spherical, for example, ellipsoidal or tetrahedral, its largest transverse dimension is equivalent to the greatest distance within the pore from one pore surface to another, e.g., the major axis length for an ellipsoidal pore or the length of the longest side for a tetrahedral pore. For those skilled in the art, one can routinely estimate the pore frequency from the average cell diameter in microns.

In one embodiment relating to vascular malformation applications and the like, to encourage cellular ingrowth and proliferation and to provide adequate fluid permeability, the average diameter or other largest transverse dimension of pores is at least about 50 μm. In another embodiment, the average diameter or other largest transverse dimension of pores is at least about 100 μm. In another embodiment, the average diameter or other largest transverse dimension of pores is at least about 150 μm. In another embodiment, the average diameter or other largest transverse dimension of pores is at least about 250 μm. In another embodiment, the average diameter or other largest transverse dimension of pores is greater than about 250 μm. In another embodiment, the average diameter or other largest transverse dimension of pores is greater than 250 μm. In another embodiment, the average diameter or other largest transverse dimension of pores is at least about 275 μm. In another embodiment, the average diameter or other largest transverse dimension of pores is greater than about 275 μm. In another embodiment, the average diameter or other largest transverse dimension of pores is greater than 275 μm. In another embodiment, the average diameter or other largest transverse dimension of pores is at least about 300 μm. In another embodiment, the average diameter or other largest transverse dimension of pores is greater than about 300 μm. In another embodiment, the average diameter or other largest transverse dimension of pores is greater than 300 μm.

In another embodiment relating to vascular malformation applications and the like, the average diameter or other largest transverse dimension of pores is not greater than about 900 μm. In another embodiment, the average diameter or other largest transverse dimension of pores is not greater than about 850 μm. In another embodiment, the average diameter or other largest transverse dimension of pores is not greater than about 800 μm. In another embodiment, the average diameter or other largest transverse dimension of pores is not greater than about 700 μm. In another embodiment, the average diameter or other largest transverse dimension of pores is not greater than about 600 μm. In another embodiment, the average diameter or other largest transverse dimension of pores is not greater than about 500 μm.

In one embodiment, the reticulated polymeric matrix that is used to fabricate the implants of this invention has any suitable bulk density, also known as specific gravity, consistent with its other properties. For example, in one embodiment, the bulk density may be from about 0.005 to about 0.15 g/cc (from about 0.31 to about 9.4 lb/ft³), preferably from about 0.015 to about 0.115 g/cc (from about 0.93 to about 7.2 lb/ft³) and most preferably from about 0.024 to about 0.104 g/cc (from about 1.5 to about 6.5 lb/ft³).

The reticulated elastomeric matrix has sufficient tensile strength such that it can withstand normal manual or mechanical handling during its intended application and during post-processing steps that may be required or desired without tearing, breaking, crumbling, fragmenting or otherwise disintegrating, shedding pieces or particles, or otherwise losing its structural integrity. The tensile strength of the starting material(s) should not be so high as to interfere with the fabrication or other processing of elastomeric matrix. Thus, for example, in one embodiment, the reticulated polymeric matrix that is used to fabricate the implants of this invention may have a tensile strength of from about 700 to about 52,500 kg/m² (from about 1 to about 75 psi). In another embodiment, elastomeric matrix may have a tensile strength of from about 7000 to about 28,000 kg/m² (from about 10 to about 40 psi). Sufficient ultimate tensile elongation is also desirable. For example, in another embodiment, reticulated elastomeric matrix has an ultimate tensile elongation of at least about 50% to at least about 500%. In yet another embodiment, reticulated elastomeric matrix has an ultimate tensile elongation of at least 75% to at least about 300%.

One embodiment for use in the practice of the invention is a reticulated elastomeric implant which is sufficiently flexible and resilient, i.e., resiliently-compressible, to enable it to be initially compressed under ambient conditions, e.g., at 25° C., from a relaxed configuration to a first, compact configuration for delivery via a delivery-device, e.g., catheter, endoscope, syringe, cystoscope, trocar or other suitable introducer instrument, for delivery in vitro and, thereafter, to expand to a second, working configuration in situ. Furthermore, in another embodiment, an elastomeric matrix has the herein described resilient-compressibility after being compressed about 5-95% of an original dimension (e.g., compressed about 19/20th-1/20th of an original dimension). In another embodiment, an elastomeric matrix has the herein described resilient-compressibility after being compressed about 10-90% of an original dimension (e.g., compressed about 9/10th-1/10th of an original dimension). As used herein, elastomeric implant has “resilient-compressibility”, i.e., is “resiliently-compressible”, when the second, working configuration, in vitro, is at least about 50% of the size of the relaxed configuration in at least one dimension. In another embodiment, the resilient-compressibility of elastomeric implant is such that the second, working configuration, in vitro, is at least about 80% of the size of the relaxed configuration in at least one dimension. In another embodiment, the resilient-compressibility of elastomeric implant is such that the second, working configuration, in vitro, is at least about 90% of the size of the relaxed configuration in at least one dimension. In another embodiment, the resilient-compressibility of elastomeric implant is such that the second, working configuration, in vitro, is at least about 97% of the size of the relaxed configuration in at least one dimension.

In another embodiment, an elastomeric matrix has the herein described resilient-compressibility after being compressed about 5-95% of its original volume (e.g., compressed about 19/20th-1/20th of its original volume). In another embodiment, an elastomeric matrix has the herein described resilient-compressibility after being compressed about 10-90% of its original volume (e.g., compressed about 9/10th-1/10th of its original volume). As used herein, “volume” is the volume swept-out by the outermost three-dimensional contour of the elastomeric matrix. In another embodiment, the resilient-compressibility of elastomeric implant is such that the second, working configuration, in vivo, is at least about 50% of the volume occupied by the relaxed configuration. In another embodiment, the resilient-compressibility of elastomeric implant is such that the second, working configuration, in vivo, is at least about 80% of the volume occupied by the relaxed configuration. In another embodiment, the resilient-compressibility of elastomeric implant is such that the second, working configuration, in vivo, is at least about 90% of the volume occupied by the relaxed configuration. In another embodiment, the resilient-compressibility of elastomeric implant is such that the second, working configuration, in vivo, occupies at least about 97% of the of volume occupied by the elastomeric matrix in its relaxed configuration.

Without being bound by any particular theory, it is believed that the absence or substantial absence of cell walls in reticulated implants when compressed to very high degree will allow them to demonstrate resilient recovery in shorter time (such as recovery time of under 15 seconds when compressed to 75% of their relaxed configuration for 10 minutes and recovery time of under 35 seconds when compressed to 90% of their relaxed configuration for 10 minutes) as compared to un-reticulated porous foams.

In one embodiment, reticulated elastomeric matrix that is used to fabricate the implants of this invention has a compressive strength of from about 700 to about 70,000 kg/m² (from about 1 to about 100 psi) at 50% compression strain. In another embodiment, reticulated elastomeric matrix has a compressive strength of from about 1,400 to about 105,000 kg/m² (from about 2 to about 150 psi) at 75% compression strain.

In another embodiment, reticulated elastomeric matrix that is used to fabricate the implants of this invention has a compression set, when compressed to 50% of its thickness at about 25° C., of not more than about 30%. In another embodiment, elastomeric matrix has a compression set of not more than about 20%. In another embodiment, elastomeric matrix has a compression set of not more than about 10%. In another embodiment, elastomeric matrix has a compression set of not more than about 5%.

In another embodiment, reticulated elastomeric matrix that is used to fabricate the implants of this invention has a tear strength, of from about 0.18 to about 1.78 kg/linear cm (from about 1 to about 10 lbs/linear inch).

In another embodiment of the invention the reticulated elastomeric matrix that is used to fabricate the implant can be readily permeable to liquids, permitting flow of liquids, including blood, through the composite device of the invention. The water permeability of the reticulated elastomeric matrix is from about 50 l/min./psi/cm² to about 500 l/min./psi/cm², preferably from about 100 l/min./psi/cm² to about 300 l/min./psi/cm². In contrast, permeability of the unreticulated elastomeric matrix is below about 1 l/min./psi/cm². In another embodiment, the permeability of the unretriculated elastomeric amtrix is below about 5 l/min./psi/cm².

In general, suitable biodurable reticulated elastomeric partially hydrophobic polymeric matrix that is used to fabricate the implant of this invention or for use as scaffold material for the implant in the practice of the present invention, in one embodiment sufficiently well characterized, comprise elastomers that have or can be formulated with the desirable mechanical properties described in the present specification and have a chemistry favorable to biodurability such that they provide a reasonable expectation of adequate biodurability.

Various biodurable reticulated hydrophobic polyurethane foams are suitable for this purpose. In one embodiment, structural materials for the inventive reticulated elastomers are synthetic polymers, especially, but not exclusively, elastomeric polymers that are resistant to biological degradation, for example, polycarbonate polyurethane-urea, polycarbonate polyurea-urethane, polycarbonate polyurethane, polycarbonate polysiloxane polyurethane, and polysiloxane polyurethane, and the like. Such elastomers are generally hydrophobic but, pursuant to the invention, may be treated to have surfaces that are less hydrophobic or somewhat hydrophilic. In another embodiment, such elastomers may be produced with surfaces that are less hydrophobic or somewhat hydrophilic.

The invention can employ, for implanting, a biodurable reticulatable elastomeric partially hydrophobic polymeric scaffold material or matrix for fabricating the implant or a material. More particularly, in one embodiment, the invention provides a biodurable elastomeric polyurethane scaffold material or matrix which is made by synthesizing the scaffold material or matrix preferably from a polycarbonate polyol component and an isocyanate component by polymerization, crosslinking and foaming, thereby forming pores, followed by reticulation of the foam to provide a biodurable reticulated elastomeric product with inter-connected and/or inter-communicating pores and channels. The product is designated as a polycarbonate polyurethane, being a polymer comprising urethane groups formed from, e.g., the hydroxyl groups of the polycarbonate polyol component and the isocyanate groups of the isocyanate component. In another embodiment, the invention provides a biodurable elastomeric polyurethane scaffold material or matrix which is made by synthesizing the scaffold material or matrix preferably from a polycarbonate polyol component and an isocyanate component by polymerization, crosslinking and foaming, thereby forming pores, and using water as a blowing agent and/or foaming agent during the synthesis, followed by reticulation of the foam to provide a biodurable reticulated elastomeric product with inter-connected and/or inter-communicating pores and channels. This product is designated as a polycarbonate polyurethane-urea or polycarbonate polyurea-urethane, being a polymer comprising urethane groups formed from, e.g., the hydroxyl groups of the polycarbonate polyol component and the isocyanate groups of the isocyanate component and also comprising urea groups formed from reaction of water with the isocyanate groups. In all of these embodiments, the process employs controlled chemistry to provide a reticulated elastomer product with good biodurability characteristics. The foam product employing chemistry that avoids biologically undesirable or nocuous constituents therein.

In one embodiment, the starting material for synthesizing the biodurable reticulated elastomeric partially hydrophobic polymeric matrix contains at least one polyol component to provide the so-called soft segement. For the purposes of this application, the term “polyol component” includes molecules comprising, on the average, about 2 hydroxyl groups per molecule, i.e., a difunctional polyol or a diol, as well as those molecules comprising, on the average, greater than about 2 hydroxyl groups per molecule, i.e., a polyol or a multi-functional polyol. In one embodiment, this soft segment polyol is terminated with hydroxyl groups, either primary or secondary. Exemplary polyols can comprise, on the average, from about 2 to about 5 hydroxyl groups per molecule. In one embodiment, as one starting material, the process employs a difunctional polyol component in which the hydroxyl group functionality of the diol is about 2. In another embodiment, the soft segment is composed of a polyol component that is generally of a relatively low molecular weight, typically from about 500 to about 6,000 daltons and preferably between 1000 to 2500 daltons. Examples of suitable polyol components include but not limited to polycarbonate polyol, hydrocarbon polyol, polysiloxane polyol, poly(carbonate-co-hydrocarbon) polyol, poly(carbonate-co-siloxane) polyol, poly(hydrocarbon-co-siloxane) polyol, polysiloxane polyol and copolymers and mixtures thereof.

In one embodiment, the starting material for synthesizing the biodurable reticulated elastomeric partially hydrophobic polymeric matrix contains at least one isocyanate component and, optionally, at least one chain extender component to provide the so-called “hard segment”. In one embodiment, the starting material for synthesizing the biodurable reticulated elastomeric partially hydrophobic polymeric matrix contains at least one isocyanate component. For the purposes of this application, the term “isocyanate component” includes molecules comprising, on the average, about 2 isocyanate groups per molecule as well as those molecules comprising, on the average, greater than about 2 isocyanate groups per molecule. The isocyanate groups of the isocyanate component are reactive with reactive hydrogen groups of the other ingredients, e.g., with hydrogen bonded to oxygen in hydroxyl groups and with hydrogen bonded to nitrogen in amine groups of the polyol component, chain extender, crosslinker and/or water. In one embodiment, the average number of isocyanate groups per molecule in the isocyanate component is about 2. In another embodiment, the average number of isocyanate groups per molecule in the isocyanate component is greater than about 2 is greater than 2.

In one embodiment, a small quantity of an optional ingredient, such as a multi-functional hydroxyl compound or other crosslinker having a functionality greater than 2, is present to allow crosslinking and/or to achieve a stable foam, i.e., a foam that does not collapse to become non-foamlike. Alternatively, or in addition, polyfunctional adducts of aliphatic and cycloaliphatic isocyanates can be used to impart crosslinking in combination with aromatic diisocyanates. Alternatively, or in addition, polyfunctional adducts of aliphatic and cycloaliphatic isocyanates can be used to impart crosslinking in combination with aliphatic diisocyanates. The presence of these components and adducts with functionality higher than 2 in the hard segment component allows for cross-linking to occur.

Exemplary diisocyanates include aliphatic diisocyanates, isocyanates comprising aromatic groups, the so-called “aromatic diisocyanates”, and mixtures thereof. Aliphatic diisocyanates include tetramethylene diisocyanate, cyclohexane-1,2-diisocyanate, cyclohexane-1,4-diisocyanate, hexamethylene diisocyanate, isophorone diisocyanate, methylene-bis-(p-cyclohexyl isocyanate) (“H12 MDI”), and mixtures thereof. Aromatic diisocyanates include p-phenylene diisocyanate, 4,4′-diphenylmethane diisocyanate (“4,4′-MDI”), 2,4′-diphenylmethane diisocyanate (“2,4′-MDI”), and mixtures thereof. Examples of optional chain extenders include diols, diamines, alkanol amines or a mixture thereof.

In one embodiment, the starting material for synthesizing the biodurable reticulated elastomeric partially hydrophobic polymeric matrix contains at least one blowing agent such as water. Other exemplary blowing agents include the physical blowing agents, e.g., volatile organic chemicals such as hydrocarbons, ethanol and acetone, and various fluorocarbons, hydrofluorocarbons, chlorofluorocarbons, and hydrochlorofluorocarbons. In one embodiment, the hard segments also contain a urea component formed during foaming reaction with water. In one embodiment, the reaction of water with an isocyanate group yields carbon dioxide, which serves as a blowing agent. The amount of blowing agent, e.g., water, is adjusted to obtain different densities of non-reticulated foams. A reduced amount of blowing agent such as water may reduce the number of urea linkages in the material.

In one embodiment, implantable device can be rendered radio-opaque to facilitate in vivo imaging, for example, by adhering to, covalently bonding to and/or incorporating into the elastomeric matrix itself particles of a radio-opaque material. Radio-opaque materials include titanium, tantalum, tungsten, barium sulfate or other suitable material known to those skilled in the art.

In one embodiment, the starting material of the biodurable reticulated elastomeric partially hydrophobic polymeric matrix is a commercial polyurethane polymers are linear, not crosslinked, polymers, therefore, they are soluble, can be melted, readily analyzable and readily characterizable. In this embodiment, the staring polymer provides a good biodurability characteristics. The reticulated elastomeric matrix is produced by taking a solution of the commercial polymer such as polyurethane and charging it into a mold that has been fabricated with surfaces defining a microstructural configuration for the final implant or scaffold, solidifying the polymeric material and removing the sacrificial mold by melting, dissolving or subliming-away the sacrificial mold. The foam product employing a foaming process that avoids biologically undesirable or nocuous constituents therein.

Of particular interest are thermoplastic elastomers such as polyurethanes whose chemistry is associated with good biodurability properties, for example. In one embodiment, such thermoplastic polyurethane elastomers include polycarbonate polyurethanes, polysiloxane polyurethanes, polyurethanes with so-called “mixed” soft segments, and mixtures thereof. Mixed soft segment polyurethanes are known to those skilled in the art and include, e.g., polycarbonate-polysiloxane polyurethanes. In another embodiment, the thermoplastic polyurethane elastomer comprises at least one diisocyanate in the isocyanate component, at least one chain extender and at least one diol, and may be formed from any combination of the diisocyanates, difunctional chain extenders and diols described in detail above. Some suitable thermoplastic polyurethanes for practicing the invention, in one embodiment suitably characterized as described herein, include: polyurethanes with mixed soft segments comprising polysiloxane together with a polycarbonate component.

In one embodiment, the weight average molecular weight of the thermoplastic elastomer is from about 30,000 to about 500,000 Daltons. In another embodiment, the weight average molecular weight of the thermoplastic elastomer is from about 50,000 to about 250,000 Daltons.

Some commercially-available thermoplastic elastomers suitable for use in practicing the present invention include the line of polycarbonate polyurethanes supplied under the trademark BIONATE® by The Polymer Technology Group Inc. (Berkeley, Calif.). For example, the very well-characterized grades of polycarbonate polyurethane polymer BIONATE® 80A, 55 and 90 are soluble in THF, DMF, DMAT, DMSO, or a mixture of two or more thereof, processable, reportedly have good mechanical properties, lack cytotoxicity, lack mutagenicity, lack carcinogenicity and are non-hemolytic. Another commercially-available elastomer suitable for use in practicing the present invention is the CHRONOFLEX® C line of biodurable medical grade polycarbonate aromatic polyurethane thermoplastic elastomers available from CardioTech International, Inc. (Woburn, Mass.).

Other possible embodiments of the materials used to fabricate the implants of this invention are described in co-pending, commonly assigned U.S. patent application Ser. No. 10/749,742, filed Dec. 30, 2003, titled “Reticulated Elastomeric Matrices, Their Manufacture and Use in Implantable Devices”, and co-pending, commonly assigned U.S. patent application Ser. No. 10/848,624, filed May 17, 2004, titled “Reticulated Elastomeric Matrices, Their Manufacture and Use In Implantable Devices” [Jones Day Docket No. 803525-999004], each of which is incorporated herein by reference in its entirely.

If desired, the reticulated elastomeric implants 36 or implants for packing the aneurysm sac, for embolizing the endoloeak nexus within the sac, for occluding the feeder vessels such as collateral arteries that drain into the aneurysm sac, or for other vascular occlusion can be rendered radio-opaque to allow for visualization of the implants in situ by the clinician during and after the procedure, employing radioimaging. Any suitable radio-opaque agent that can be covalently bound, adhered or otherwise attached to the reticulated polymeric implants may be employed including without limitation, tantalum and barium sulfate. In addition to incorporating radiopaque agents such as tantalum into the implant material itself, a further embodiment of the invention encompasses the use of radio-opaque metallic components to impart radiopacity to the implant. For example, thin filaments comprised of metals with shape memory properties such as platinum or nitinol can be embedded into the foam implant and may be in the form of a straight or curved wire, helical or coil-like structure, umbrella structure, or other structure generally known to those skilled in the art. Alternatively, a metallic frame around the implant may also be used to impart radiopacity. The metallic frame may be in the form of a tubular structure similar to a stent, a helical or coil-like structure, an umbrella structure, or other structure generally known to those skilled in the art. Attachment of radiopaque metallic components to the implant can be accomplished by means including but not limited to chemical bonding or adhesion, suturing, pressure fitting, compression fitting, and other physical methods.

Some optional embodiments of the invention comprise apparatus or devices and treatment methods employing biodurable reticulated elastomeric implants 36 into which biologically active agents are incorporated for the matrix to be used for controlled release of pharmaceutically-active agents, such as a drug, and for other medical applications. Any suitable agents may be employed as will be apparent to those skilled in the art, including, for example, but without limitation thrombogenic agents, e.g., thrombin, anti-inflammatory agents, and other therapeutic agents that may be used for the treatment of abdominal aortic aneurysms. The invention includes embodiments wherein the reticulated elastomeric material of the implants is employed as a drug delivery platform for localized administration of biologically active agents into the aneurysm sac. Such materials may optionally be secured to the interior surfaces of elastomeric matrix directly or through a coating. In one embodiment of the invention the controllable characteristics of the implants are selected to promote a constant rate of drug release during the intended period of implantation.

The implants with reticulated structure with sufficient and required liquid permeability and permit blood, or other appropriate bodily fluid, to access interior surfaces of the implants, which optionally are drug-bearing. This happens due to the presence of inter-connected, reticulated open pores that form fluid passageways or fluid permeability providing fluid access all through and to the interior of the matrix for elution of pharmaceutically-active agents, e.g., a drug, or other biologically useful materials.

Implants 36 for packing the aneurysm sac or implants for embolizing the endoloeak nexus within the sac and occluding the feeder vessels such as collateral arteries that drain into the aneurysm sac desirably have microstructural interior surfaces, which may be described as “endoporous” surfaces in the case of reticulated implants, which surfaces are compatible with endothelialization, the attachment and proliferation of cells that can lead to formation of endothelial tissue. In one embodiment, hydrophobic or partially hydrophobic biocompatible, and preferably biodurable, polymeric materials are believed satisfactory for this purpose when employed with a suitable matrix morphology that permits blood or other bodily fluids access to the surfaces during the process of endothelialization.

In another embodiment, the matrix of the reticulated elastomeric implants 36 may, for example, be endoporously hydrophilized, as described hereinabove, by post treatments or by setting the elastomer in a hydrophilic environment, to render its microstructural surfaces chemically more reactive. If desired, biologically useful compounds, or controlled release formulations containing them, may be attached to the endoporous surfaces for local delivery and release some possibilities for which are described in the following co-pending, commonly assigned U.S. patent applications: U.S. patent application Ser. No. 10/749,742, filed Dec. 23, 2003, entitled “Reticulated Elastomeric Matrices, Their Manufacture and Use in Implantable Devices”, and U.S. patent application Ser. No. 10/692,055, filed Oct. 22, 2003, entitled “Method and System for Intra-Vesicular Delivery of Therapeutic Agents”, each of which is incorporated herein by reference in its entirety.

In a further embodiment of the invention, the pores biodurable reticulated elastomeric matrix that are used to fabricate the implants of this invention are coated or filled with a cellular ingrowth promoter. In another embodiment, the promoter can be foamed. In another embodiment, the promoter can be present as a film. The promoter can be a biodegradable material to promote cellular invasion of pores biodurable reticulated elastomeric matrix that are used to fabricate the implants of this invention in vivo. Promoters include naturally occurring materials that can be enzymatically degraded in the human body or are hydrolytically unstable in the human body, such as fibrin, fibrinogen, collagen, elastin, hyaluronic acid and absorbable biocompatible polysaccharides, such as chitosan, starch, fatty acids (and esters thereof), glucoso-glycans and hyaluronic acid. In some embodiments, the pore surface of the biodurable reticulated elastomeric matrix that are used to fabricate the implants of this invention is coated or impregnated, as described in the previous section but substituting the promoter for the biocompatible polymer or adding the promoter to the biocompatible polymer, to encourage cellular ingrowth and proliferation.

The invention also provides an apparatus and methods for delivering one or more biodurable elastomeric reticulated and resilient, polymeric implants to a target vascular site for the treatment and prevention of endoleaks. One embodiment of the invention involves distal loading of the implant into the tip of a delivery catheter using a loader apparatus. Essentially, four steps are required, namely, compression of the implant, loading of the implant into the delivery catheter, tracking of the delivery catheter through an introducer or guide sheath which has been positioned in the vascular system at the target site, and ejection of the implant out of the delivery catheter. The invention consists of a loader apparatus for compressing and loading the implant, a split delivery catheter for introduction to the target vascular site, and an obturator or pusher for ejection of the implant.

The steps of compressing and loading the implant into the split delivery catheter can be achieved through use of mechanical force as applied with the loader apparatus as shown in FIG. 7. The loader apparatus of the invention consists of a main body 130, knob 132, and plunger 134. The internal mechanism as shown in FIG. 8 consists of a stainless steel band 136, slide 138, and lead screw 140. The implant is pre-loaded into a cartridge or holding mechanism which maintains the implant in a relaxed, uncompressed state 142.

To compress the implant, the knob 132 is rotated thereby turning the lead screw 140 and enabling the slide 138 to move and pull the stainless steel band 136. This application of mechanical force causes the band to circumferentially reduce the diameter of the implant in the cartridge 142. The use of mechanical force is critical to fully compress the implant from its initial, relaxed state, to a final, compressed state which can fit within the lumen of the delivery catheter. The final implant size is reached when the slide 138 reaches a fixed stopping point. The plunger 134 is then depressed enabling the transfer and loading of the compressed implant from the loader cartridge into the tip or distal end of the split delivery catheter, which is placed and held in a hole located opposite the plunger 134.

Substantial or even moderate compression of the implant may result in significant frictional force resisting discharge from the loader apparatus into the delivery catheter. Usefully, to mitigate the friction, the cartridge or holding mechanism 142 which contains the implant can be highly polished and/or coated or formed of a low-friction material such as silicone or polytetrafluoroethylene.

The split delivery catheter of the invention is shown in FIG. 9 and consists of a sheath 144 with a slit down the length of the sheath 146, a tapered front end 148, a hemostasis bypass sleeve 150, and a handle 152. Preferably, the split delivery catheter is made of a strong biocompatible material such as high density polyethylene or is of a braided design, to provide strength necessary to navigate through tortuous vessels with minimal kinking and maximum trackability. After the implant is loaded into the tip of the split delivery catheter 148 using the loader apparatus, the delivery catheter is removed from the loader apparatus. The hemostasis bypass sleeve 150 is slid from its proximal position near the handle 152 approximately 1-2 mm past the split end of the delivery catheter tip 148. This action closes the taper of the delivery catheter tip 148, secures the implant in place in the tip of the delivery catheter, and allows the delivery catheter to slide easily past the valve of an introducer or guide sheath which has been previously placed in the vascular system of the patient.

Suitable introducer or guide sheaths are known to the art and can range in size from 5 Fr to about 14 Fr, preferably no more than about 9 Fr. Some, but not all, desirable embodiments of the invention employ catheters of about 6 Fr to 7 Fr. After passage of the split delivery catheter of the invention through the introducer valve, the hemostasis bypass sleeve 150 is pulled back to its starting position at the proximal position near the handle 152. The split delivery catheter is then advanced through the lumen of the introducer until the hemostasis bypass sleeve 150 rests against the introducer hub. At this point, the tip of the split delivery catheter 148 is aligned with the tip of the introducer sheath.

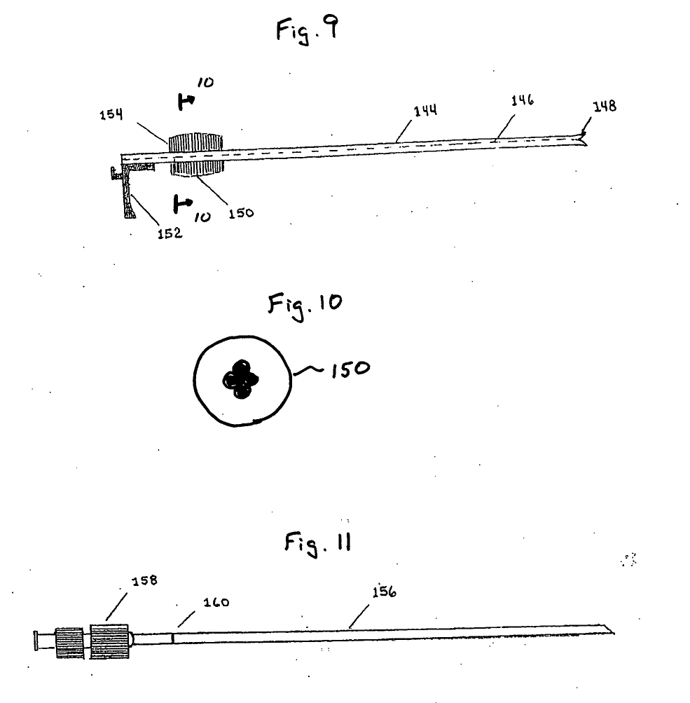

The proximal end of the hemostasis bypass sleeve 150 has a “keyed” back end as shown in FIG. 10. When the split delivery catheter of the invention is rotated ¼ turn, the handle of the catheter 152 serves as a “key” to enable the delivery catheter to be pushed forward. At this point, the implant which is still contained in the tip of the split delivery catheter 148 is now positioned outside the introducer sheath and is ready for deployment. Substantial compression of the implant may result in significant frictional force resisting discharge from the delivery catheter. Usefully, when the delivery catheter is pushed beyond the tip of the introducer sheath, the split end of the catheter 148 opens up having been released from the constraints of the introducer sheath, thereby reducing the frictional force on the implant and facilitating ejection of the implant into the vasculature.

The obturator or pusher of the invention is shown in FIG. 11 and consists of a metallic shaft 156, a handle 158, and a marker 160. The obturator shaft 156 can be comprised of various materials including but not limited to high- and low-density polyethylene and metals such as stainless steel, nitinol, and titanium. A preferred embodiment is a metallic material which provides advantages including kink-resistance, strength, and radiopacity. Once the split delivery catheter has been advanced through the introducer sheath and the implant is ready for deployment into the target site, the obturator is introduced into the lumen of the split delivery catheter until the marker 160 on the obturator shaft 156 is lined up with the handle of the delivery catheter 152. This position indicates that the end of the obturator is aligned against the proximal end of the compressed implant which is still contained in the tip of the delivery catheter 148. The handle of the obturator 158 can now be pushed forward until it is flush against the handle of the delivery catheter, thereby ejecting the implant out of the delivery catheter into the target vascular site.

In another embodiment, the implant can be delivered by introducing a compressed implant into the proximal end of a guide catheter for subsequent pushing or advancement through the entire length of the catheter and discharging from the distal end using an obturator. The steps of compressing and loading the implant into the guide catheter can be achieved through use of a plastic or metal funnel or loader in which the implant is forced through decreasing cross-section and then introduced through the valve of the guide catheter. The reduction in cross-section of the plastic or metal loader can be gradual and continuous or in steps. Alternatively, the implant can be hand-rolled or compressed into a hemostasis bypass sleeve which is then used to puncture the valve of the guide catheter. Subsequent to the introduction of the compressed foam implant into the proximal end of the guide catheter, an obturator can be used to advance to compressed foam through the length of the catheter and to discharge the implant out the distal end into the target vascular site.

The delivery apparatus of the invention can be used to deliver one or multiple implants into the aneurysm sac or other target volume using methods generally known to those skilled in the art. For example, a direct translumbar injection or puncturing method may be employed in which a needle is used to penetrate through the patient's skin, followed by introduction of an introducer sheath, guide sheath, or guide catheter through the needle. The implant can then be delivered through the introducer sheath, guide sheath, or guide catheter, by using the distal loading method, loader device, and split delivery catheter, or by using the proximal introduction method and introduction devices, as described herein.

An alternative method for advancing an introducer to the target site comprises transarterial delivery with percutaneous access or percutaneous delivery. In this alternative method an introducer or guide sheath can be advanced from a femoral artery access point to the desired position in the aneurysm sac or other target vascular site. If the target site is the aneurysm sac, the introducer can be advanced into the space between an implanted endograft and an adjacent blood vessel wall once the endograft has been deployed. If the target site is a feeder vessel which is a source of endoleaks, including but are not limited to, lumbar arteries, the inferior mesenteric artery, and the internal iliac arteries, the introducer can be advanced from a femoral artery access point to the target vessel through methods known to those skilled in the art. The implant can then be delivered through the introducer sheath, guide sheath, or guide catheter, by using the distal loading method, loader device, and split delivery catheter, or by using the proximal introduction method and introduction devices, as described herein.

Bulk volume reduction of the implants for delivery can be facilitated by further implant embodiments of the invention which complement the application of mechanical force by the loader apparatus. In one embodiment, achieving substantial or maximum bulk volume reduction is desirable to enable filling of the target vascular volume with the smallest number of implants so as to reduce the number of catheterization cycles. One embodiment involves elongation of the implants within the loader apparatus and delivery catheter, for example by stretching, twisting, or stretching and twisting, giving the implants elongated configurations well adapted for accommodation in a suitable delivery catheter. Without being bound by any particular theory, the elastomeric nature of the reticulated implant material (with its associated of Poisson's ratio) will lead to reduction in the thickness of the solid struts when the implant is stretched or twisted or subjected to both, thereby creating additional volume that can be compressed to obtain higher compression ratio in the implant. This will allow for delivery of larger implants.

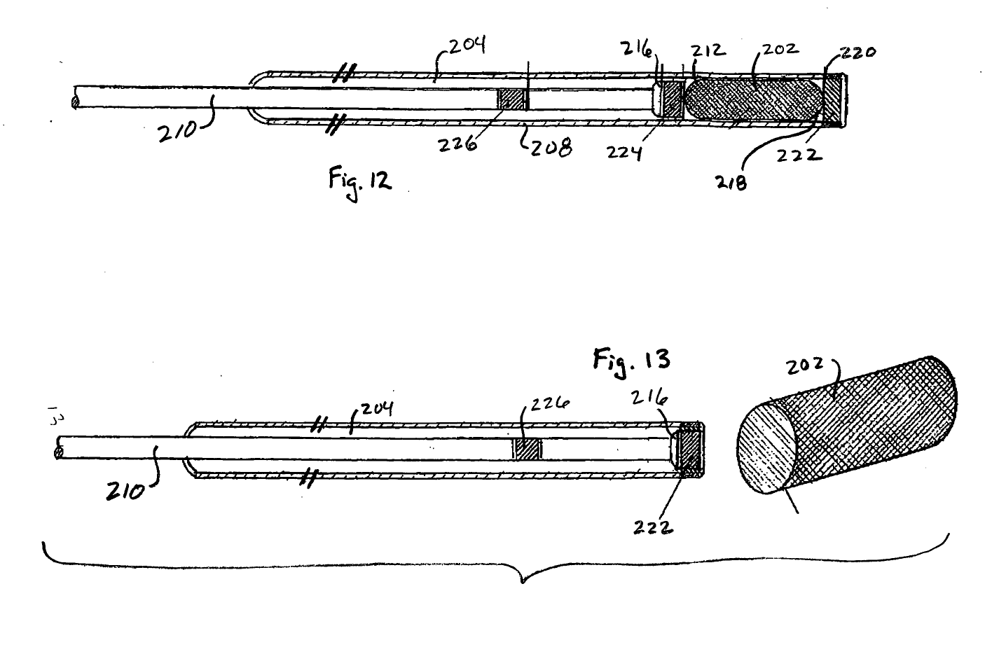

In a preferred device for delivering an implant, as shown in FIG. 12, an implant 202 is introduced into a lumen 204 of the proximal end (not shown) of a sheath or catheter 208, and a pusher rod or obturator 210 advances implant 202 through lumen 204 of catheter 208. A compressed implant 202 is positioned within lumen 204 at the distal portion 212 of catheter 208, with the distal tip 216 of obturator 210 positioned adjacent to the proximal portion 218 of implant 202. The distal tip 220 of catheter 208 has a radio-opaque marker 222. Preferably a radio-opaque marker 224 is positioned at obturator distal tip 216, and another radio-opaque marker 226 is positioned proximal to marker 224 to indicate implant positioning, preferably at a distance from marker 224 comparable to the length of implant 202.

When all three radio-opaque markers 222, 224, and 226 are visible on x-ray or ultrasound spaced equidistantly, that means that implant 202 is located at the tip of catheter 208, ready for deployment. This is helpful, “alert” information for the operator to have. Controlled deployment can be accomplished by slow advancement of implant 202, watching radio-opaque markers 222 and 224 and allowing enough time for the foam of implant 202 to recover to full volume. The change of distance between markers 222 and 224 will indicate how much of implant 202 is still in catheter 208 and how much has been ejected. When, as shown in FIG. 13, obturator 202 is moved distally to eject implant 202 (shown expanded), radio-opaque markers 222 and 224 align to indicate to the operator that implant 202 has been ejected. Optionally contrast could be injected distally through the obdurator 210 to support recovery of the foam in implant 202 by pressurizing the foam while it is still partially in catheter 208.

Another embodiment involves the use of hollow implants, so selected to enhance compressibility while still permitting implants to resist blood flow. The hollow interior volumes of the implants can constitute any suitable proportion of the respective implant volume, for example in the range of about 10 percent to about 90 percent, with other useful volumes being in the range of about 30 percent to about 50 percent. Such implants in an expanded, uncompressed state can be compressible by a factor from about 2:1 up to about 10:1 and more preferably from 3:1 to 4.9:1.

The invention provides for one or more delivery catheters, loaded or repeatedly loaded, if necessary, with sufficient implants to constitute a desired group of implants for treatment of a target vascular site. To effect delivery, the implants can be manually loaded into the delivery catheter by the clinician using the apparatus and methods described above. Alternatively, the implants can be preloaded into the tip of an implant delivery catheter supplied with the implants “on board”. Suitable catheters to accommodate one or two implants each, and possibly more, are known and other suitable catheters that become available subsequently to this application may also be employed. Optionally, where a large number of catheterization cycles are required to deliver a group of implants, the cycle of catheter loading with one or more implants, advance of the catheter, ejection of the one or more implants in a desired manner at the target site, and retraction of the catheter ready for reloading may be mechanized or automated. Alternatively, the implants can be contained in a bioabsorbable sheath or shrink-wrapped, in a compressed state, which sheath or shrink-wrapped package is easily loaded into the delivery catheter or introducer sheath and delivered to the target site. At the target site, the sheath or package may be hydrolyzed or otherwise eroded in situ, for example in the course of about 6 to about 72 hours, to release the implants which then expand into the volume at the target site.

Another embodiment of the invention relates to an alternate implant positioning procedure. Initially a guide catheter is advanced to position the distal tip of the guide catheter near or adjacent to a targeted site in a patient's vasculature using a standard delivery technique. Next, to accomplish optimal stretching and compression of the foam for the delivery position, an implant is pull-inserted from a fully expanded position at the proximal end of an introducer instrument by using a string with a knot that is attached to the implant and extends to and out from the distal end of the introducer. The implant is slowly pulled into the distal area of the introducer instrument until the knot advances past the distal end of the introducer. A blade or scalpel is used to sever the string, including the knot, as close to the knot as possible, at the tip of the introducer instrument. The blade or scalpel is then used to push excess foam back into introducer distal tip, until it remains completely inside.

The introducer loaded with the implant is inserted directly into the hub of the guide catheter, or the side arm is used to stabilize the connection and straighten alignment of both lumens. A plunger is used to introduce the implant by pushing from the proximal end of the introducer completely into the guide catheter lumen using the total length of the plunger. After the plunger is withdrawn, a pusher is introduced into the guide catheter through a side port.

The implant is then advanced toward the radio-opaque marker on the distal tip of the guide catheter using the pusher or obturator. The radio-opaque marker on the distal tip of the pusher enables the physician to monitor implant positioning within the guide catheter. Advancement of the implant is stopped when the marker on the pusher indicates that the implant is approximately 70% to 90% deployed out of the catheter tip (visible distance). Optionally a two marker system on the pusher can be used to provide more precise distance control during implant deployment. Contrast media is then slowly injected through the lumen of the hollow pusher or obturator while the implant is partially deployed, serving to fill the implant with contrast media. This method of partial deployment of the implant serves two purposes. First, partial deployment facilitates full implant recovery and vessel occlusion by pressurizing the implant with the contrast media. Secondly, partial deployment enables a slow, controlled delivery which minimizes the risk of distal emblization or migration of the implant, which might occur while the implant is not yet fully recovered.

After total occlusion is confirmed, the rest of the implant should be deployed from the guide catheter. The pusher should be removed so that a final angiogram can be performed.

One aspect of the invention provides for the treatment of late, or post-operative endoleaks that are identified after an endograft has been implanted, for example one month up to two years after deployment. The existence of such late endoleaks can be identified in post-operative computerized tomography, “CT” scans that are generally performed at regular intervals following an endograft procedure. Pursuant to the present invention, one method of treating late endoleaks comprises the introduction of an occupying body of individual, shaped implants into the aneurysm sac or in the feeding vessel responsible for the endoleak(s). The occupying body of implants can be selected to fill the proportion of the aneurysm sac in the perigraft space occupied by the endoleak(s) in order to reduce blood flow and thereby reduce the hemodynamic forces acting on the aneurysm or other vascular wall. To effect delivery, the implants can be loaded into the tip of the implant delivery catheter in a compressed state. The loaded implant delivery catheter can then be advanced through the lumen of an introducer sheath, guide sheath, or guide catheter having a distal end or tip which is appropriately positioned within the aneurysm sac or other target internal patient volume, for example a volume in the vasculature. Once the implant delivery catheter is advanced through the introducer sheath to the desired position in the aneurysm sac or other target site, the reticulated elastomeric implant can be deployed. Alternatively, the implant can be delivered by introducing a compressed implant into the proximal end of a guide catheter for subsequent pushing or advancement through the entire length of the catheter and discharging from the distal end using an obturator. Introduction of the implant into the guide catheter can be achieved by using a plastic or metal funnel or loader or a hemostasis bypass sleeve. Subsequent to the introduction of the compressed foam implant into the proximal end of the guide catheter, an obturator can be used to advance and discharge the implant into the target vascular site. In one preferential embodiment of the invention, such treatment will only occur in the presence of an expanding aneurysm sac.

Another aspect of the invention provides for the prevention of endoleaks which can arise following endovascular repair by prophylactically implanting a suitable number of reticulated elastomeric implants at the time of performing the endovascular repair procedure. Pursuant to the present invention, one method of endoleak prevention comprises catheter-based introduction of a plurality of implants into the endograft perigraft space, after the endograft has been deployed but before the procedure is completed. While it is desirable to minimize the number of implants and thus catheterization cycles, it is not feasible to put in a few large foam implants due to the technical barriers associated with compressing and delivering large implants through the lumens of introducers commonly used in such procedures, which range in size from 4 to 9 Fr but are more preferably in the range of 5 to 7 Fr. Larger sized or larger diameter catheters or introducers have a problem of accessing the target endoleak sites especially in the presence of the endograft. This smaller sized catheters or introducers are necessitated by the extreme difficulty and formidable challenge in delivering a few large implants through a long narrow or small diameter catheter. The endoleak treatment sites are difficult to access owing to narrow passage and lack of maneuverability in the space surrounding the pre-existing endograft or the endgraft that is put in prior to the implants being inserted for prophylactic or peri-operative treatments for endovascular problems.

In such a prophylactic method of the invention, the implants can be delivered through an introducer sheath, guide sheath, or guide catheter, by using the distal loading method, loader device, and split delivery catheter, or by using the proximal introduction method and introduction devices, as described herein. After the main body of the endograft is deployed, but before termination of the endograft deployment procedure, the implants can be delivered through the lumen of an introducer sheath, guide sheath, or guide catheter which has been appropriately positioned within the sac. The implants can be deployed using an obturator or pusher to expel the implants from the catheter distal tip into the perigraft space in the aneurysm sac or other target volume.

In general, it is desirable for the biodurable reticulated elastomeric implants employed for packing the aneurysm sac, occluding side branches or feeder vessels and for other associated endoleak treatment pursuant to the invention to be substantially oversized with respect to the introducer instrument which can, for example, be a delivery catheter. The implants can usefully be compressed by any suitable factor, for example, to have an effective diameter smaller than the effective diameter of a delivery instrument, such as a factor of at least about 2:1, preferably up to about 4.3:1. In another embodiment, the implants can be usefully compressed up to a ratio of about 5.8:1 or even higher. The compression factor refers to the uncompressed to compressed ratio of one dimension of the implant in the direction of compression, for example the cross-sectional radius or diameter of a cylindrical implant. For example, for a nominally solid cylindrical implant formed of a reticulated elastomeric material having a 96% void volume, the radial compression is about 4.9× meaning that the uncompressed diameter is about 4.9 times the compressed radius. High degrees of compression can be useful in implementing the inventive methods, by reducing the number of iterations of catheterization that are required to fill a given target volume. In one embodiment, implants with diameters smaller than the diameter of a delivery instrument can also be delivered.

Some considerations limiting the degree of compression it is desirable to utilize in practice include the effect on the force required to discharge a compressed implant from the introducer and possible effects upon the volume recoverability of the implant. Some useful embodiments of the invention compress implants 36 into an introducer for delivery to the target site to a degree of from about 1.5:1 to about 10:1 referring to the proportion of the relaxed volume to the compressed volume respectively. Particularly useful are degrees of compression in the range of from about 2:1 to about 4.8:1.

The invention includes methods and a device and delivery apparatus for the treatment of an aneurysm or other vascular defect which requires embolization or occlusion to stop undesirable blood flow or perfusion. The invention includes selecting one or more reticulated elastomeric implants to fill or occlude a target vascular site, loading the occupying body of implants under compression into the distal end of a suitable introducer instrument, and deploying such implants to the target vascular site whereby such implants achieve occlusion through mechanisms including thrombosis, fibrosis, and endothelialization.

The following examples demonstrate aspects of the invention:

### EXAMPLE 1

### Fabrication of a Crosslinked Reticulated Polyurethane Matrix

The aromatic isocyanate RUBINATE 9258 (from Huntsman) was used as the isocyanate component. RUBINATE 9258, which is a liquid at 25° C., contains 4,4′-MDI and 2,4′-MDI and has an isocyanate functionality of about 2.33. A diol, poly(1,6-hexanecarbonate)diol (POLY-CD CD220 from Arch Chemicals) with a molecular weight of about 2,000 Daltons was used as the polyol component and was a solid at 25° C. Distilled water was used as the blowing agent. The blowing catalyst used was the tertiary amine triethylenediamine (33% in dipropylene glycol; DABCO 33LV from Air Products). A silicone-based surfactant was used (TEGOSTAB® BF 2370 from Goldschmidt). A cell-opener was used (ORTEGOL® 501 from Goldschmidt). The viscosity modifier propylene carbonate (from Sigma-Aldrich) was present to reduce the viscosity. The proportions of the components that were used are set forth in the following table:

TABLE 1

| Ingredient | Parts by Weight |
|---|---|
| Polyol Component | 100 |
| Viscosity Modifier | 5.80 |
| Surfactant | 0.66 |
| Cell Opener | 1.00 |
| Isocyanate Component | 47.25 |
| Isocyanate Index | 1.00 |
| Distilled Water | 2.38 |
| Blowing Catalyst | 0.53 |

The polyol component was liquefied at 70° C. in a circulating-air oven, and 100 g thereof was weighed out into a polyethylene cup. 5.8 g of viscosity modifier was added to the polyol component to reduce the viscosity, and the ingredients were mixed at 3100 rpm for 15 seconds with the mixing shaft of a drill mixer to form “Mix-1”. 0.66 g of surfactant was added to Mix-1, and the ingredients were mixed as described above for 15 seconds to form “Mix-2”. Thereafter, 1.00 g of cell opener was added to Mix-2, and the ingredients were mixed as described above for 15 seconds to form “Mix-3”. 47.25 g of isocyanate component were added to Mix-3, and the ingredients were mixed for 60±10 seconds to form “System A”.

2.38 g of distilled water was mixed with 0.53 g of blowing catalyst in a small plastic cup for 60 seconds with a glass rod to form “System B”.

System B was poured into System A as quickly as possible while avoiding spillage. The ingredients were mixed vigorously with the drill mixer as described above for 10 seconds and then poured into a 22.9 cm×20.3 cm×12.7 cm (9 in.×8 in.×5 in.) cardboard box with its inside surfaces covered by aluminum foil. The foaming profile was as follows: 10 seconds mixing time, 17 seconds cream time, and 85 seconds rise time.

Two minutes after the beginning of foaming, i.e., the time when Systems A and B were combined, the foam was placed into a circulating-air oven maintained at 100-105° C. for curing for from about 55 to about 60 minutes. Then, the foam was removed from the oven and cooled for 15 minutes at about 25° C. The skin was removed from each side using a band saw. Thereafter, hand pressure was applied to each side of the foam to open the cell windows. The foam was replaced into the circulating-air oven and postcured at 100-105° C. for an additional four hours.

The average pore diameter of the foam, as determined from optical microscopy observations, was greater than about 275 μm.

The following foam testing was carried out according to ASTM D3574: Bulk density was measured using specimens of dimensions 50 mm×50 mm×25 mm. The density was calculated by dividing the weight of the sample by the volume of the specimen. A density value of 2.81 lbs/ft³ (0.0450 g/cc) was obtained.

Tensile tests were conducted on samples that were cut either parallel to or perpendicular to the direction of foam rise. The dog-bone shaped tensile specimens were cut from blocks of foam. Each test specimen measured about 12.5 mm thick, about 25.4 mm wide, and about 140 mm long; the gage length of each specimen was 35 mm and the gage width of each specimen was 6.5 mm. Tensile properties (tensile strength and elongation at break) were measured using an INSTRON Universal Testing Instrument Model 1122 with a cross-head speed of 500 mm/min (19.6 inches/minute). The average tensile strength perpendicular to the direction of foam rise was determined as 29.3 psi (20,630 kg/m²). The elongation to break perpendicular to the direction of foam rise was determined to be 266%. The measurement of the liquid flow through the material is measured in the following way using a iquid permeability apparatus or Liquid Permeaeter (Porous Materials, Inc., Ithaca, N.Y.). The foam sample was 8.5 mm in thickness and covered a hole 6.6 mm in diameter in the center of a metal plate that was placed at the bottom of the Liquid Permeaeter filled with water. Thereafter, the air pressure above the sample was increased slowly to extrude the liquid from the sample and the permeability of water through the foam was determined to be 0.11 L/min/psi/cm².

### EXAMPLE 2

### Reticulation of a Crosslinked Polyurethane Foam

Reticulation of the foam described in Example 1 was carried out by the following procedure: A block of foam measuring approximately 15.25 cm×15.25 cm×7.6 cm (6 in.×6 in.×3 in.) was placed into a pressure chamber, the doors of the chamber were closed, and an airtight seal to the surrounding atmosphere was maintained. The pressure within the chamber was reduced to below about 100 millitorr by evacuation for at least about two minutes to remove substantially all of the air in the foam. A mixture of hydrogen and oxygen gas, present at a ratio sufficient to support combustion, was charged into the chamber over a period of at least about three minutes. The gas in the chamber was then ignited by a spark plug. The ignition exploded the gas mixture within the foam. The explosion was believed to have at least partially removed many of the cell walls between adjoining pores, thereby forming a reticulated elastomeric matrix structure.

The average pore diameter of the reticulated elastomeric matrix, as determined from optical microscopy observations, was greater than about 275 μm. A scanning electron micrograph image of the reticulated elastomeric matrix of this example (not shown here) demonstrated, e.g., the communication and interconnectivity of pores therein.

The density of the reticulated foam was determined as described above in Example 1. A post-reticulation density value of 2.83 lbs/ft³ (0.0453 g/cc) was obtained.

Tensile tests were conducted on reticulated foam samples as described above in Example 1. The average post-reticulation tensile strength perpendicular to the direction of foam rise was determined as about 26.4 psi (18,560 kg/m²). The post-reticulation elongation to break perpendicular to the direction of foam rise was determined to be about 250%. The average post-reticulation tensile strength parallel to the direction of foam rise was determined as about 43.3 psi (30,470 kg/m²). The post-reticulation elongation to break parallel to the direction of foam rise was determined to be about 270%.

Compressive tests were conducted using specimens measuring 50 mm×50 mm×25 mm. The tests were conducted using an INSTRON Universal Testing Instrument Model 1122 with a cross-head speed of 10 mm/min (0.4 inches/minute). The post-reticulation compressive strengths at 50% compression, parallel to and perpendicular to the direction of foam rise, were determined to be 1.53 psi (1,080 kg/m²) and 0.95 psi (669 kg/m²), respectively. The post-reticulation compressive strengths at 75% compression, parallel to and perpendicular to the direction of foam rise, were determined to be 3.53 psi (2,485 kg/m²) and 2.02 psi (1,420 kg/m²), respectively. The post-reticulation compression set, determined after subjecting the reticulated sample to 50% compression for 22 hours at 25° C. then releasing the compressive stress, parallel to the direction of foam rise, was determined to be about 4.5%.

The resilient recovery of the reticulated foam was measured by subjecting 1 inch (25.4 mm) diameter and 0.75 inch (19 mm) long foam cylinders to 75% uniaxial compression in their length direction for 10 or 30 minutes and measuring the time required for recovery to 90% (“t-90%”) and 95% (“t-95%”) of their initial length. The percentage recovery of the initial length after 10 minutes (“r-10”) was also determined. Separate samples were cut and tested with their length direction parallel to and perpendicular to the foam rise direction. The results obtained from an average of two tests are shown in the following table:

TABLE 2

| Time | | | | |
|---|---|---|---|---|
| compressed | Test Sample | t-90% | t-95% | r-10 |
| (min) | Orientation | (sec) | (sec) | (%) |
| 10 | Parallel | 6 | 11 | 100 |
| 10 | Perpendicular | 6 | 23 | 100 |
| 30 | Parallel | 9 | 36 | 99 |
| 30 | Perpendicular | 11 | 52 | 99 |

In contrast, a comparable foam with little to no reticulation typically has t-90 values of greater than about 60-90 seconds after 10 minutes of compression.

The measurement of the liquid flow through the material is measured in the following way using a Liquid permeability apparatus or Liquid Permeaeter (Porous Materials, Inc., Ithaca, NY). The foam samples were between 7.0 and 7.7 mm in thickness and covered a hole 8.2 mm in diameter in the center of a metal plate that was placed at the bottom of the Liquid Permeaeter filled with water. The water was allowed to extrude through the sample under gravity and the permeability of water through the foam was determined to be 180 L/min/psi/cm² in the direction of foam rise and 160 L/min/psi/cm² in the perpendicular to foam rise.

### EXAMPLE 3

### Fabrication of a Crosslinked Polyurethane Matrix

The isocyanate component was RUBINATE 9258, as described in Example 1. A polyol comprising 1,6-hexamethylene polycarbonate (Desmophen LS 2391, Bayer Polymers), i.e., a diol, with a molecular weight of about 2,000 Daltons was used as the polyol component and was a solid at 25° C. Distilled water was used as the blowing agent. The blowing catalyst, surfactant, cell-opener and viscosity modifier of Example 1 were used. The proportions of the components that were used is set forth in the following table:

TABLE 3

| Ingredient | Parts by Weight |
|---|---|
| Polyol Component | 150 |
| Viscosity Modifier | 8.72 |
| Surfactant | 3.33 |
| Cell Opener | 0.77 |
| Isocyanate Component | 81.09 |
| Isocyanate Index | 1.00 |
| Distilled Water | 4.23 |
| Blowing Catalyst | 0.67 |

The polyol component was liquefied at 70° C. in a circulating-air oven, and 150 g thereof was weighed out into a polyethylene cup. 8.7 g of viscosity modifier was added to the polyol component to reduce the viscosity and the ingredients were mixed at 3100 rpm for 15 seconds with the mixing shaft of a drill mixer to form “Mix-1”. 3.3 g of surfactant was added to Mix-1 and the ingredients were mixed as described above for 15 seconds to form “Mix-2”. Thereafter, 0.77 g of cell opener was added to Mix-2 and the ingredients were mixed as described above for 15 seconds to form “Mix-3”. 81.09 g of isocyanate component was added to Mix-3 and the ingredients were mixed for 60±10 seconds to form “System A”.

4.23 g of distilled water was mixed with 0.67 g of blowing catalyst in a small plastic cup for 60 seconds with a glass rod to form “System B”.

System B was poured into System A as quickly as possible while avoiding spillage. The ingredients were mixed vigorously with the drill mixer as described above for 10 seconds then poured into a 22.9 cm×20.3 cm×12.7 cm (9 in.×8 in.×5 in.) cardboard box with its inside surfaces covered by aluminum foil. The foaming profile was as follows: 11 seconds mixing time, 22 seconds cream time, and 95 seconds rise time.

Two minutes after the beginning of foaming, i.e., the time when Systems A and B were combined, the foam was place into a circulating-air oven maintained at 100-105° C. for curing for 1 hour. Thereafter, the foam was removed from the oven and cooled for 15 minutes at about 25° C. The skin was removed from each side using a band saw and hand pressure was applied to each side of the foam to open the cell windows. The foam was replaced into the circulating-air oven and postcured at 100-105° C. for additional 4 hours and 30 minutes.

The average pore diameter of the foam, as determined from optical microscopy observations, was about 247 μm.

The density of the foam was determined as described in Example 1. A density value of 2.9 lbs/ft³ (0.046 g/cc) was obtained.

The tensile properties of the foam were determined as described in Example 1. The tensile strength, determined from samples that were cut perpendicular to the direction of foam rise, was 24.64±2.35 psi (17,250±1,650 kg/m²). The elongation to break, determined from samples that were cut perpendicular to the direction of foam rise, was 215±12%.

Compressive tests were conducted as described in Example 2. The compressive strength, determined from samples that were cut parallel to the direction of foam rise at 50% compression, was 1.74±0.4 psi (1,225±300 kg/m²). The compression set, determined from samples that were cut parallel to the direction of foam rise after subjecting the samples to 50% compression for 22 hours at 40° C. then releasing the compressive stress, was about 2%.

The tear resistance strength of the foam was conducted as described in Example 2. The tear strength was determined to be 2.9±0.1 lbs/inch (1.32±0.05 kg/cm).

The pore structure and its inter-connectivity were characterized using a Liquid Extrusion Porosimeter (Porous Materials, Inc., Ithaca, N.Y.). In this test, the pores of a 25.4 mm diameter cylindrical sample 4 mm thick were filled with a wetting fluid having a surface tension of about 19 dynes/cm then that sample was loaded into a sample chamber with a microporous membrane, having pores about 27 μm in diameter, placed under the sample. Thereafter, the air pressure above the sample was increased slowly to extrude the liquid from the sample. For a low surface tension wetting fluid, such as the one used, the wetting liquid that spontaneously filled the pores of the sample also spontaneously filled the pores of the microporous membrane beneath the sample when the pressure above the sample began to increase. As the pressure continued to increase, the largest pores of the sample emptied earliest. Further increases in the pressure above the sample led to the empting of increasingly smaller sample pores as the pressure continued to increase. The displaced liquid passed through the membrane and its volume was measured. Thus, the volume of the displaced liquid allowed the internal volume accessible to the liquid, i.e., the liquid intrusion volume, to be obtained. The liquid intrusion volume of the foam was determined to be 4 cc/g.

The measurement of the liquid flow through the material is measured in the following way using a Liquid permeability apparatus or Liquid Permeaeter (Porous Materials, Inc., Ithaca, N.Y.). The foam sample was 7.5 mm in thickness and covered a hole 6.5 mm in diameter in the center of a metal plate that was placed at the bottom of the Liquid Permeaeter filled with water. Thereafter, the air pressure above the sample was increased slowly to extrude the liquid from the sample and the permeability of water through the foam was determined to be 0.54 L/min/psi/sqcm.

### EXAMPLE 4

### Reticulation of a Crosslinked Polyurethane Foam

Reticulation of the foam described in Example 1 was carried out by the procedure described in Example 2.

Tensile tests were conducted on reticulated foam samples as described in Example 2. The density of the reticulated foam was determined as described in Example 1. A post-reticulation density value of 2.46 lbs/ft³ (0.0394 g/cc) was obtained.

The post-reticulation tensile strength, measured on samples that were cut perpendicular to the direction of foam rise, was about 20 psi (14,080 kg/m²). The post-reticulation elongation to break, measured on samples that were cut perpendicular to the direction of foam rise, was about 189%.

Compressive tests of the reticulated foam were conducted as described in Example 2. The post-reticulation compressive strength, measured on samples that were cut parallel to the direction of foam rise, at 50% and 75% compression, was about 1.36 psi (957 kg/m²) and about 2.62 psi (1,837 kg/m²), respectively.

The tear resistance strength of the foam was conducted as described in Example 2. The tear strength was determined to be 2.6 lbs/inch (1.2 kg/cm).

The pore structure and its inter-connectivity are characterized using a Liquid Extrusion Porosimeter as described in Example 2. The liquid intrusion volume of the reticulated foam was determined to be 28 cc/g and the permeability of water through the reticulated foam was determined to be 184 L/min/psi/sqcm as described in Example 2. These results demonstrate, e.g., the interconnectivity and continuous pore structure of the reticulated foam.

The resilient recovery of the reticulated foam subjected to 75% uniaxial compression for 10 or 30 minutes was measured by the method described in Example 2, subjecting 1 inch (25.4 mm) diameter and 0.75 inch (19 mm) long foam cylinders and measuring the time required for recovery to 90% (“t-90%”) and 95% (“t-95%”) of their initial length. The percentage recovery of the initial length after 10 minutes (“r-10”) was also determined. Separate samples were cut and tested with their length direction parallel to and perpendicular to the foam rise direction. The results are set forth in the following table:

TABLE 4

| Time | | | | |
|---|---|---|---|---|
| Compressed | Test Sample | t-90% | t-95% | r-10 |
| (min) | Orientation | (sec) | (sec) | (%) |
| 10 | Parallel | 6 | 11 | 100 |
| 10 | Perpendicular | 6 | 23 | 100 |
| 30 | Parallel | 9 | 36 | 99 |
| 30 | Perpendicular | 11 | 52 | 99 |

In contrast, a comparable foam with little to no reticulation typically has t-90 values of greater than about 60-90 seconds after 10 minutes of compression.

### EXAMPLE 5

### Implant Ability to Provide Resistance and Cause Reduction in Fluid Flow Rates Through Simulated Body Conduits

This example demonstrates how implants, with varying dimensions and shapes and subjected to different compression ratios, will provide resistance and cause consequent reduction in liquid flow in channels or conduits subjected to a range of pressures corresponding to systolic pressure levels experienced in humans. The tests were conducted in a flow model system model designed to simulate typical vessels that transport blood in the body and the system model was designed and fabricated to simulate physiologic flow and pressure experienced in humans.

Vessels were simulated using synthetic vascular grafts (or conduits) made from PTFE with a diameter of 6 mm. This graft size was chosen to mimic the typical target vessel diameter that is intended to be targeted for occlusion or embolization, e.g., internal iliac artery. The pressure range was chosen to mimic the systolic pressure experienced by these vessels in actual human applications.

The flow model system is comprised of a perfusion pump, surgical tubing, connectors, c-clamps, conduit, and a reservoir. The pump provides constant flow rate while the c-clamps provide the necessary resistance to simulate physiologic pressure. Two pressure sensors were placed at the entry and exit to the graft conduit to measure the pressure drop. Hespan® (supplied by American Hospital Supply Corporation) was chosen as the test medium to for its similar viscosity to that of human blood.

The material for preparing the implant was prepared following the method described in Example 4 and the implants were machine cut into desired shapes and size. Implants were placed into the PTFE grafts using an introducer system. Flow pressures through the conduit were increased from 50 mmHg through 250 mm Hg in increments of 50 mmHg. The pressure and flow data (average of two runs) were collected and presented in the following table:

TABLE 5
Flow Measurement of Hespan ® through 6-mm Vascular Graft under
Varying Pressures Following Deployment of Different Implant
Configurations.

| | | | Radial | | |
|---|---|---|---|---|---|
| Vascular | | Implant | Com- | | Flow |
| Graft | | Dimensions - | pression | Flow | Rate |
| Diameter | Implant | Diameter × Length | Strain | Pressure | (cc/ |
| (mm) | Shape | (mm) | (%) | (mmHg) | min) |
| 6 mm | Cylin- | 10 mm D × 10 mm L | 40 | 100 | 16 |
| | der | | | 250 | 24 |
| | | 12 mm D × 10 mm L | 50 | 100 | 12 |
| | | | | 250 | 18 |
| 6 mm | Tapered | 10 mm D × 10 mm L | 40 | 100 | 17 |
| | Cylin- | | | 250 | 36 |
| | der | 12 mm D × 10 mm L | 50 | 100 | 6 |
| | | | | 250 | 18 |

The flow rate of Hespan®, unimpeded by any implant in the graft or conduit, was approximately 600 cc/min.

Significant reduction in flow was thus obtained in all runs by placing the implants in the 6 mm diameter conduit. The data above also demonstrates that the flow through the conduit can be controlled by varying the implant size and implants that are compressed more, offer higher flow resistances, i.e, larger sized implants led to lower flow rates when placed in the same sized conduits. These results show that under the normal systolic pressure, experienced by blood carrying vessels in humans, the implants of this invention will provide an immediate resistance to the flow of body fluid, such as blood and the decreased blood flow rate (together with activation of a coagulation cascade and thrombotic response at least partially as a response to this reduced blood flow) should lead to the formation of a clot. In time initiate the process by which cellular entities such as fibroblasts and tissues can invade and grow into the reticulated elastomeric implants, creating a biological occlusion.

### EXAMPLE 6

### Efficacy of Plurality of Implants Introduced in the Perigraft Space and a Delivery Method to Treat Endoleaks Following AAA Endograft Implantation in a Canine Model

An in-vivo experiment was conducted to validate the efficacy of adjunctive treatment of the aneurysm sac to prevent and treat endoleaks. The implants were cut from the material prepared following the method described in Example 4 and the implant configuration was a double tapered cylinder measuring 10 mm diameter by 20 mm length. The implants were sterilized using gamma irradiation at a dosage level of 25 kilograys.

The success of endovascular repair of abdominal aortic aneurysms (AAA) is dependent on exclusion of the aneurysm from the arterial circulation as incomplete exclusion exposes the aneurysm wall to systemic arterial pressure. Intra-aneurysmal pressure is transmitted to the aneurysm wall and may lead to continued aneurysm expansion and a significant risk of rupture and death. In this experiment, multiple foam implants were used to pack the sac of a canine abdominal aortic aneurysm (AAA) to determine the effects on intra-aneurysmal pressure, as described below.

A canine AAA model was developed to measure the effectiveness of endovascular treatments for AAA. A prosthetic infrarenal aneurysm was surgically created in a canine model by grafting a 4×4 cm Dacron patch with an attached solid-state pressure transducer over a longitudinal arteriotomy in the abdominal aorta below the renal arteries and above the aortic bifurcation. The pressure transducer enabled the physician to measure intra-aneurysmal pressure or IAP, defined as the pressure on the aneurysmal vessel wall from any blood flow within the perigraft space in the sac. The caudal mesenteric artery and the multiple lumbar arteries were left intact to generate persistent type II retrograde endoleaks. The transducer cable was tunneled subcutaneously to exit between the scapulae. The aneurysms were left in place for two weeks to allow for healing of the aortic suture line.

In a second radiological procedure, a WL Gore ViaBahn stent-graft measuring 8 mm×5 cm was deployed into the vascular system from a femoral access point into the aneurysm using an introducer sheath. Once the stent-graft was secured in place and was determined through angiography to have excluded the aneurysm sac, a 9 Fr 30 cm Cook Check-Flo introducer sheath was deployed into the vascular system from a femoral access point. The introducer sheath was advanced into the perigraft space within the sac by proceeding into the space between the implanted stent-graft and the adjacent blood vessel wall.

Following successful positioning of the Cook introducer sheath within the perigraft space of the sac, a custom-made 30 cm 8 Fr catheter, made from low-density polyethylene with a split in its distal delivery, was advanced through the hemostasis valve of the Cook introducer sheath until the handle of the split delivery catheter resisted further advancement. Prior to insertion of the split delivery catheter into the Cook introducer sheath, one foam implant in the configuration of a double tapered cylinder had been loaded into the tip of the split delivery catheter by manual compression using disposable Adson stainless steel 4¾″ forcepsOnce the split delivery catheter was fully advanced within the Cook introducer sheath, a custom-made obturator comprised of polyethylene was introduced into the lumen of the split delivery catheter and used to eject or deploy the foam implant into the perigraft space of the aneurysm.

After ejection of the foam implant, the delivery catheter was withdrawn and used to reload another foam implant into the tip of the catheter. After loading, the delivery catheter was re-introduced into the Cook introducer sheath, after which the obturator was re-introduced into the lumen of the split delivery catheter to deploy the foam implant. Consecutive implant loading and delivery cycles were repeated until the physician felt resistance for delivering additional implants, and thereby determined that the sac was fully packed. An angiogram was taken to confirm angiographic occlusion of the perigraft space by the foam implants. The Cook introducer sheath was maintained in place for the duration of the consecutive implant loading and delivery cycles, to keep the number of catheterization cycles to one.

A total of four dogs were treated with foam implants and compared to four control animals with no side branches and no endoleaks. Periodically both intra aneurysmal pressure and systemic pressures were monitored. Type II endoleaks generated considerable intraaneurysmal pressurization that was significantly reduced from systemic pressure (P<0.001) as shown in Table 6 below. Untreated Type II endoleaks result in intraaneurysmal pressures that average 70/%-80% of systemic pressure. Treatment with polyurethane foam induced thrombosis of the endoleak and feeding arteries in all four animals. It resulted in nearly complete elimination of intra aneurysmal pressure (P<0.001) making it indistinguishable from control aneurysms with no endoleaks (P=NS). Cine MRA, Duplex and angiography documented persistent patency up to the time of euthanasia (mean, 64 days) for untreated type II endoleaks and confirmed thrombosis of polyurethane treated endoleaks.

TABLE 6
Pressure Measurements of Treatment and Control Animals in an
Established Canine Model of AAA Endoleaks

| | Systolic | Mean | Pulse | Endoleak |
|---|---|---|---|---|
| | Pressure* | Pressure* | Pressure* | Patency |
| Patent Type II | 0.702 | 0.784 | 0.406 | Patent |
| Endoleak | | | | |
| Polyurethane Treated | 0.183 | 0.142 | 0.054 | Thrombosed |
| Type II Endoleak | | | | |
| Control (No Endoleak/ | 0.172 | 0.137 | 0.089 | Thrombosed |
| No Branches) | | | | |
| Systemic Pressure | 1.0 | 1.0 | 1.0 | NA |
| P-Value (Patent vs. | <0.001 | <0.001 | <0.001 | <0.001 |
| Polyurethane Treated) | | | | |

*All pressures listed were measured after antegrade AAA exclusion and are indexed as a percentage the systemic pressure.

The results demonstrate the thrombosis of endoleaks by polyurethane foam implants occurs rapidly and results in near abolition of intra aneurysmal pressure. The experiment validates the utility of reticulated, porous, resilient implants for the prevention/treatment of endoleaks.

### EXAMPLE 7

### Healing and Biological Tissue Response of a Single Implant Placed in the Carotid Artery of the Rabbit

An in-vivo experiment was conducted to evaluate the biological tissue response to a single, oversized occlusive implant surgically placed in the carotid artery of the rabbit. The implants were cut from the material prepared following the method described in Example 4 and the implant configuration was a cylinder measuring 3 mm diameter by 10 mm length. The implant had been sterilized using gamma irradiation at a dosage level of 25 kilograys.

A rabbit model was used in which a single foam implant was placed in the carotid artery via direct surgical implantation. There were three groups of rabbits with three animals each group (n=3). One group of rabbits was sacrificed at each of three timepoints post-surgery: 24 hours, 2 weeks, and 4 weeks. The primary endpoint of the study was histologic description of the tissue response to the implant.

The rabbits were anesthetized. The hair was clipped and the skin was prepared for aseptic surgery. A skin incision was made over the right carotid artery. Following soft tissue dissection and isolation of the artery, an arteriotomy was performed and the foam implant was placed in the vessel proximal (i.e., closer to the heart) to the arteriotomy. The arteriotomy site was closed. The subcutaneous tissue and skin were closed with sutures. There were no complications of implant placement.

All nine animals survived until their study respective study endpoints when they were euthenized and the artery of interest with the embedded implant was removed. The tissues were trimmed, embedded in paraffin, and sectioned at six micron thickness. The tissues were stained with Hematoxylin and Eosin (H&E) and Masson's trichrome stain if necessary for histological evaluation. The frozen sections were stained for vWF to evaluate the presence and distribution of endothelial cells.

All vessels into which the test article was placed showed total occlusion of blood flow immediately following placement of the implant and closure of the arteriotomy site. All of the implanted carotid arteries appeared grossly occluded and atretic at the time of explant.

Vessels in which the implant were placed showed ingrowth of host cells onto the surface of the implant. The cells present in the 24-hour group consisted of a mixture of polymorphonuclear leukocytes and mononuclear cells. The cells present in the two-week and four-week groups consisted exclusively of mononuclear cells and spindle shaped cells consistent with endothelial cells that appeared to grow along the struts of the porous implant. Occasional blood-filled channels were noted in the two-week and four-week groups. These blood-filled channels were lined by endothelial cells.

The vessel wall immediately adjacent to the occluded lumen of the vessel showed accumulations of mononuclear inflammatory cells and spindle cells that were consistent with fibroblasts/fibrocytes. The number of mononuclear cells and spindle cells was very small in the 24-hour group but prominent in the two-week and four-week groups.

The subjacent muscular layers of the occluded arteries maintained their three-dimensional architecture and showed no evidence of degeneration, necrosis, or inflammation in any of the three groups.

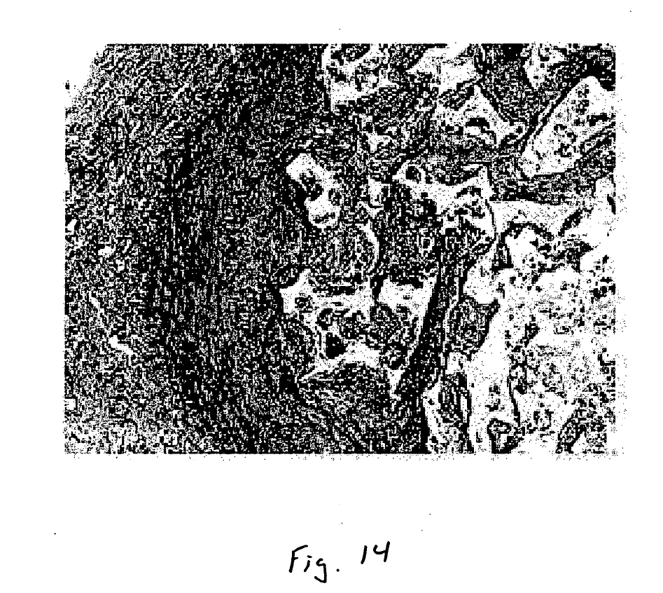

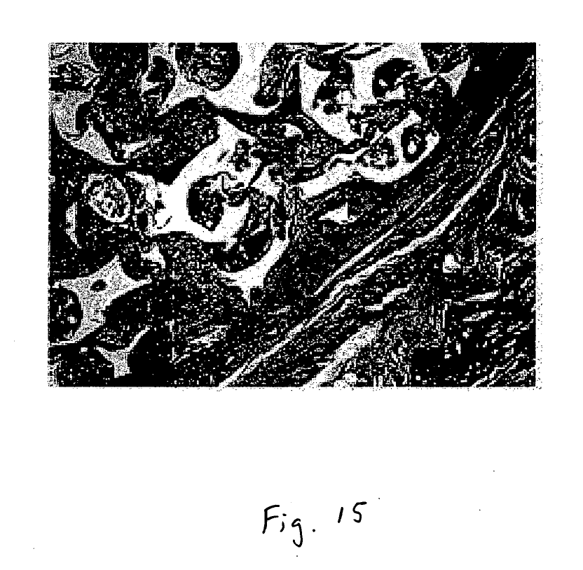

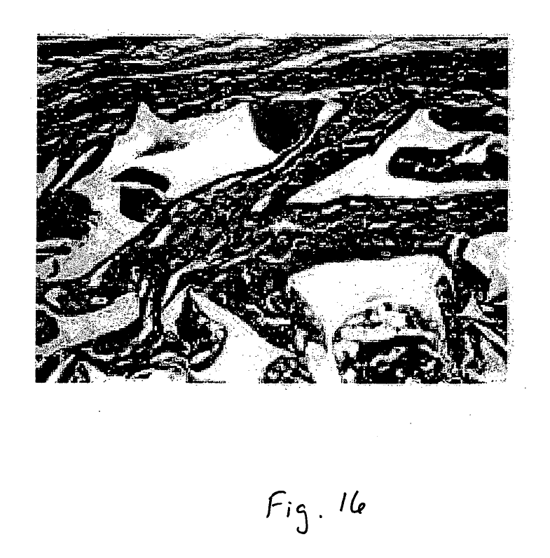

The implants were effective in causing biological occlusion in all vessels in which it was placed. FIGS. 14, 15, and 16 show the biological occlusive response. The host response consisted of small amounts of fibrous connective tissue and mononuclear inflammatory cells. More particularly, FIG. 14 is a 20× magnification of a treated vessel wall that shows an intact muscular layer (red staining) and integrated contact surface between a vessel lumen and an implant. Blood-filled structures, i.e., vessels, are noted within the porous structure of the implant. FIGS. 15 and 16 are two representative images (20× and 40×, respectively) of the interface between vessel wall and implant. Cell nuclei and connective tissue can be seen interspersed with the implant in the lumen of the vessel. Blood-filled capillary-like structures can also be noted within the lines of the implant.

This study demonstrated that the implants functioned as a totally occlusive barrier to blood flow in the arteries in which it was placed. The host-tissue response to the implants was consistent with the expected mammalian response, specifically, small amounts of fibrous connective tissue with a low-grade mononuclear cell response.

### EXAMPLE 8

### Acute and Short-Term Occlusion Efficacy of Foam Implants Delivered Percutaneously in a Swine Peripheral Embolization Model

An in-vivo experiment using percutaneously delivered foam implants was conducted to (i) validate implant deliverability using a custom-made loader and split catheter delivery system via a “front-end” loading approach, (ii) verify implant oversizing requirements, and (iii) verify acute and short-term occlusion efficacy in a swine peripheral embolization model. Implants were cut from the material prepared following the method described in Example 4 and the implant configuration was a double tapered cylinder measuring 6 mm diameter by 15 mm length. The implant had been sterilized using gamma irradiation at a dosage level of 25 kilograys.

To deliver the implants, a surgical cutdown in the carotid was first performed following standard practices for vessel puncture and access. A 9F Terumo Introducer Set was utilized to secure access to the carotid artery. A Cook 7 F 90 cm Flexor® Check-Flo® Introducer sheath was then advanced to the target site over a guidewire. After positioning the introducer at the target site, the guidewire was withdrawn, leaving only the introducer in place.

The foam implant was then loaded into the custom-made loader device as follows. First, the access cap of the loader was removed and the implant was placed inside the cylinder formed by the steel band of the loader. The access cap was then placed back on the loader. The black knob at the end of the loader was turned clockwise until it reached a complete stop, thereby compressing the implant to its target diameter for insertion into the delivery catheter split at the tip called spilt delivery catheter. After implant compression, the loader was placed on the operating table so that the access cap was positioned towards the operator's right and the delivery system alignment hub was positioned towards the operator's left. The plunger was placed into the hole in the access cap until it came into contact with the compressed implant. On the opposing side of the access cap, the distal end of the split delivery catheter was placed into the delivery system alignment hub of the loader. Prior to placing the split delivery catheter into the delivery system alignment hub, the hemostasis bypass sleeve was previously positioned just proximal to the split end of the delivery catheter. While holding the distal end of the split delivery catheter firmly in place, the plunger was depressed, thereby ejecting the implant into the distal tip of the split delivery catheter. The hemostasis bypass sleeve was then slid distally until it contacted the delivery system alignment hub of the loader. The split delivery catheter was then withdrawn from the delivery system alignment hub of the loader into the hemostasis bypass sleeve, thereby enveloping the loaded tip of the split delivery catheter inside the hemostasis bypass sleeve.

The implant was then deployed into the target vascular site as follows: The split delivery catheter with the implant loaded into the split tip was introduced into the Cook introducer sheath, using the bypass sleeve to penetrate the valve of the introducer sheath. The split delivery catheter was progressed forward by 2 cm, and then the hemostasis bypass sleeve was pulled back out of the introducer valve. Hemostasis was thereby achieved on the split delivery catheter. The split delivery catheter was then pushed through the introducer sheath until the proximal connector rested against the hemostasis bypass sleeve. This indicated that the distal tip of the split delivery catheter was lined up with the distal tip of the introducer sheath. The hub of the split delivery catheter was rotated approximately 1/4 turn and pushed forward, such that the hub was fully seated in the keyed back end of the hemostasis bypass sleeve. This indicated that the implant was located just distal to the tip of the introducer sheath and was ready for deployment. To deploy the implant out of the split delivery catheter into the vessel, the back end of the implant was pushed by an obturator thereby deploying the implant into the target vascular site.

Five different vessels ranging in size from 3.0 mm to 5.5 mm were occluded with five double tapered implants each measuring 6 mm diameter×15 mm length using a custom-made loader, split delivery catheter, and obturator via a “front-end” delivery approach. This procedure was successfully repeated in five different vessels, including segments of the femoral artery, external iliac artery, common iliac artery, and common carotid artery. These vessels were sequentially occluded with a single implant in each vessel following the procedure outlined above. All five implants were successfully delivered using the custom delivery system and “front-end” loading procedure described above, thereby validating percutaneous delivery of elastomeric implants using this approach.

An angiogram was performed 45 seconds to 1 minute following implant deployment to verify acute occlusion efficacy. All vessels demonstrated angiographic occlusion, thereby verifying acute occlusion efficacy of percutaneously delivered elastomeric implants. Vessel diameters ranged from 3.0 mm to 5.5 mm. Based on these target vessel diameters, it was determined that implant oversizing of 10% to 100% successfully results in vessel occlusion. This animal was sacrificed acutely.

### EXAMPLE 9

### Short-Term Occlusion Efficacy of Foam Implants Delivered Percutaneously in a Swine Peripheral Embolization Model

Following the same procedure as outlined in Example 8, a single 6 mm×15 mm implant also made in a similar fashion as in Example 8 was delivered percutaneously via a “front-end” loading approach using the custom-made loader, split delivery catheter, and obturator, into a target vascular site in the external ilio-femoral artery. The animal was sacrificed after one week. Followup angiographic analysis at one week indicated that the vessel was 100% occluded (no recanalization). Histology analysis supported total occlusion of the vascular site.

This in-vivo experiment validated percutaneous delivery of an elastomeric implant via a “front-end” loading approach using a custom-made compression and delivery system. The study also verified acute and short-term occlusion efficacy of elastomeric implants with target oversizing of as little as 10% (defined as oversizing of the implant diameter to the target vessel diameter). Following the same procedure as outlined in example 8, a single 6 mm×15 mm implant also made in a similar fashion as in example 8 was delivered percutaneously via a “front-end” loading approach using the custom-made loader, split delivery catheter, and obturator, into a target vascular site in the external ilio-femoral artery. Stainless steel embolization coils (Cook Inc.) were placed in the contralateral artery to serve as controls. The animal was sacrificed after 1 week. Followup angiographic analysis at 1 week indicated that the foam implant vessel was 100% occluded (no recanalization) vs. 50-60% recanalization of the coil vessel.

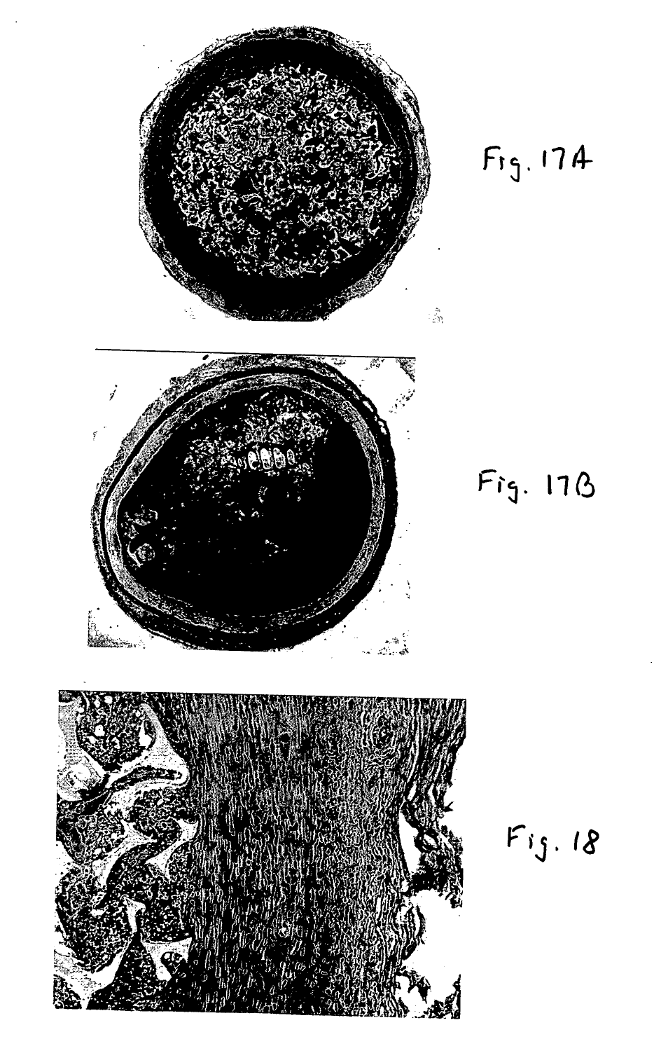

Histology analysis supported total occlusion of the vascular site by the foam implant, with minimal inflammatory response, no necrosis of the perivascular tissues, biological integration with the vessel wall, and cellular infiltration into the structure of the reticulated implant. In contrast, the coil control demonstrated severe damage to the vessel wall (arterial perforation), with minimal biological occlusion or cell ingrowth. FIGS. 17A and 17B show the histological contrast between the foam implant (FIG. 17A) vs. coils (FIG. 17B) at one week. FIG. 18 shows the cellular infiltration and vessel wall adherence engendered by the foam implant by one week.

This in-vivo experiment validated percutaneous delivery of an elastomeric implant via a “front-end” loading approach using a custom-made compression and delivery system. The study also verified angiographic and biological occlusion superiority of elastomeric implants in comparison to the current standard-of-care, coils.

### EXAMPLE 10

### Evaluation of Percutaneously Delivered Foam Implants vs. Stainless Steel Coils in a Swine Peripheral Embolization Model

An in-vivo experiment using percutaneously delivered foam implants was conducted to (i) validate implant deliverability using a custom-made hemostasis bypass sleeve via a “back-end” loading approach, (ii) compare acute procedural outcomes for foam implants vs. the current standard-of-care for percutaneous embolization, stainless steel coils, and (iii) compare followup angiographic occlusion outcomes for foam implants vs. coils through one month. Implants were cut from the material prepared following the method described in Example 2 and the implant configuration was a double tapered cylinder measuring 6 mm diameter by 15 mm length. The implant had been sterilized using gamma irradiation at a dosage level of 25 kilograys.

Animals were implanted with either foam implants or stainless steel coils, as necessary, to cause angiographic occlusion of the ilio-femoral segment. A total of twenty-eight (28) swine underwent the procedure with either the foam implants (n=22) or coils (n=6). In the foam implant arm, implants measuring 6 mm diameter×15 mm length were deployed in 3-5 mm vessel segments. In the coil control arm, Cook Embolization Coils ranging from 3-5 mm diameter and 2-5 cm length were deployed in 3-5 mm vessel segments as necessary to cause angiographic occlusion. Animals were sacrificed at one week and one month. Endpoints included time-to-occlusion, implant migration following deployment, procedural time and angiographic occlusion at followup.

To deliver the foam implants, a surgical cutdown in the carotid was first performed following standard practices for vessel puncture and access. A 9Fr Cook Introducer Set was utilized to secure access to the carotid artery. A Cook 7 Fr 90 cm Flexor® Check-Flo® Introducer sheath was then advanced to the target site over a guidewire. After the introducer was positioned at the target site, the guidewire was withdrawn, leaving only the introducer in place.

The foam implant was then loaded into the hemostasis bypass sleeve as follows. The implant was wetted in sterile saline. The implant was manually compressed by gentle rolling and then insertion into the metal tube of the hemostasis bypass sleeve.

The foam implant was deployed into the target vascular site as follows. The introducer sheath was flushed with sterile saline. The metal tube of the hemostasis bypass sleeve was then inserted into the valve of the introducer sheath's hemostasis valve. An obturator was used to push the implant out of the hemostasis bypass sleeve into the introducer sheath. The obturator was used to continue to push the implant through the length of the introducer and out the tip into the target vascular site, thereby deploying the foam implant. An angiogram was performed in one-minute increments following deployment of the implant to confirm angiographic occlusion.

The coils were delivered into the control animals as per manufacturer instructions-for-use (Cook Inc).

One foam implant was used in each of the 22 test animals. An average of four stainless steel coils were used in each of the six control animals. The acute procedural outcomes from this experiment are shown in the Table 7 below. The foam implant arm shows superior acute procedural outcomes vs. coil controls in terms of shorter time-to-occlusion, reduced distal migration, and minimized procedural time.

TABLE 7
Acute Procedural Outcomes for Foam Implants vs. Cook Embolization
Coils in a Swine Peripheral Embolization Model.

| Study | Sample | Occlusion | Migration | Procedural |
|---|---|---|---|---|
| Arm | Size | Time (min) | (mm) | Time (hrs) |
| Biomerix Vascular | n = 22 | 1.68 + 0.70 min | 0.20 + 0.55 mm | 0.88 + 0.24 hrs |
| Occlusion Device | | | | |
| Cook Embolization | n = 6 | 5.83 + 1.60 min | 40.83 + 78.38 mm | 1.25 + 0.44 hrs |
| Coils | | | | |
| P-value | — | p < 0.001 | p = 0.02 | p = 0.01 |

Angiographic occlusion at the one-week and one-month sacrifices are shown in the following table:

TABLE 8

| | | Angiographic | Angiographic |
|---|---|---|---|
| | | occlusion success | occlusion success |
| Study Arm | Sample Size | at 1 week⁽¹⁾ | at 1 month⁽¹⁾ |
| Biomerix | N = 6/timepoint | 83% (5/6) | 100% (6/6) |
| Vascular | | | |
| Occlusion | | | |
| Device | | | |

| | | |
|---|---|---|
| Cook | N = 2/timepoint | 75% (3/4)⁽²⁾ |
| Embolization | | |
| Coils | | |

⁽¹⁾Occlusion success is defined as 90% + angiographic occlusion.
⁽²⁾Results were combined due to small sample size.

These results support superior acute procedural outcomes and angiographic occlusion outcomes through 1 month for a novel reticulated porous polymer implant vs. the current standard of care, coils. The experiment also validates implant deliverability using a custom-made hemostasis bypass sleeve via a “back-end” loading approach.

### EXAMPLE 11

### Radial Compression of the Implant

The foams were investigated for quantifying the minimum diameter to which they could be compressed for delivery through a catheter. Foams were made as per Example 4 and machined into cylindrical implants with diameter of 6 mm and 15 mm in length.

These implants could be compressed to an average diameter of 1.35 mm (n=4) when the axis of the cylindrical implant was parallel to the foam rise direction and to an average diameter of 1.40 mm (n=4) when the axis of the cylindrical implant was perpendicular to the foam rise direction. This translates to the fact that the diameter of the foam implants could be compressed by approximately 78% and 77%, when the axis of the cylindrical implant was parallel and perpendicular to the foam rise direction, respectively.

### EXAMPLE 12

### Radio-Opaque Formulation of Cross-Linked Biodurable Foam

A radio-opaque formulation of a cross-linked biodurable foam was made using procedures similar to those described in Example 1 with the following proportions of the components as shown in the following table:

TABLE 9

| Ingredient | Parts by Weight |
|---|---|
| Polyol Component (Poly CD(TM) CD220 | 100 |
| Viscosity Modifier (Propylene carbonate) | 5.80 |
| Tantalum nanoparticle powder (Aldrich) | 12.67 |
| Surfactant (Tegostab BF 2370) | 0.66 |
| Cell Opener (Ortegol 501) | 1.00 |
| Isocyanate Component (Rubinate 9258) | 47.25 |
| Isocyanate Index | 1.00 |
| Distilled Water | 2.43 |
| Blowing Catalyst (Dabco 33 LV) | 0.53 |

The foaming profile was as follows: 10 seconds mixing time, 16 seconds cream time, and 76 seconds rise time. The radio-opaque member was initially mixed as a part of System A.

Two minutes after the beginning of foaming, i.e., the time when Systems A and B were combined, the foam was place into a circulating-air oven maintained at 102° C. for curing for 50 minutes. Thereafter, the foam was removed from the oven and cooled for 15 minutes at about 25° C. The skin was removed from each side using a band saw and hand pressure was applied to each side of the foam to open the cell windows. The foam was replaced into the circulating-air oven and postcured at 100° C. for additional 3 hours.

The average pore diameter of the foam, as determined from optical microscopy observations, was about 310 μm.

The density of the foam was determined as described in Example 1. A density value of 2.83 lbs/ft³ (0.045 g/cc) was obtained.

The tensile properties of the foam were determined as described in Example 1. The tensile strength, determined from samples that were cut parallel to the direction of foam rise, was 38.9 psi (27,400 kg/m²). The elongation to break, determined from samples that were cut parallel to the direction of foam rise, was 238%.

Compressive tests were conducted as described in Example 2. The compressive strength, determined from samples that were cut parallel to the direction of foam rise at 50% compression, was 2.0 psi (1,410 kg/m²) and at 75% compression was 4.4 psi (3070 kg/m²).

Reticulation process described in Example 2 can be used to reticulate the foam.

The preceding specific embodiments are illustrative of the practice of the invention. It is to be understood, however, that other expedients known to those skilled in the art or disclosed herein, may be employed without departing from the spirit of the invention or the scope of the appended claims.

1. A device for treating or preventing a vascular condition at a mammalian vascular site, which comprises an implant formed from a compressible elastomeric matrix in a shape conducive to delivery through a delivery instrument.

2. The device of claim 1, wherein the matrix comprises reticulated, interconnected and intercommunicating networks of voids and/or pores to permit ingrowth of tissue.

3. The device of claim 1, wherein the device has a major effective diameter of from about 0.5 mm to about 100 mm.

4. The device of claim 3, wherein the device has a major effective diameter of from about 1 mm to about 20 mm.

5. The device of claim 1, wherein each implant comprises a biodurable, reticulated elastomeric matrix.

6. The device of claim 5, wherein the matrix is a polycarbonate polyurethane-urea, polycarbonate polyurea-urethane, polycarbonate polyurethane, or polycarbonate polysiloxane polyurethane.

7. The device of claim 1, wherein the matrix is cross-linked.

8. The device of claim 1, wherein the matrix is thermoplastic.

9. The device of claim 1, wherein the matrix is compressible and resiliently recoverable.

10. The device of claim 1, wherein the matrix is biocompatible.

11. The device of claim 1, wherein the matrix is at least partially hydrophobic.

12. The device of claim 1, wherein the structural matrix has a hydrophilic surface treatment or a hydrophilic coating.

13. The device of claim 1, wherein the implant has a shape selected from the group consisting of cylindrical, cylindrical with hollow center, cylindrical with an annulus, conical, frustoconical, single tapered cylindrical, double tapered cylindrical, bullet-shaped, ring-shaped, C-shaped, S-shaped spiral, helical, spherical, spherical with hollow center, spherical with hollow not at the center, spherical with slits, elliptical, ellipsoidal, polygonal, star-like, rods, cubic, pyramidal, tetrahedronal, trapezoidal, parallelepiped, ellipsoidal, fusiform, tubular, sleeve-like, folded, coiled, helical, and compounds or combinations of two or more of the foregoing.

14. The device of claim 13, wherein the implant is cylindrical, bullet-shaped, and/or tapered on one or both ends.

15. The device of claim 1 which has a metallic frame.

16. The device of claim 15, wherein the frame comprises a shape memory metal.

17. The device of claim 1 which comprises a radio-opaque agent or structural element.

18. The device of claim 17, wherein the agent is tantalum or barium sulfate.

19. The device of claim 17, wherein the structural element comprises platinum, nitinol, titanium, or gold.

20. The device of claim 1 which comprises a biologically active agent.

21. A system for treating or preventing a vascular condition at a mammalian vascular site, which comprises:
one or more compressible implants comprising biodurable reticulated elastomeric matrix, and
a delivery instrument into which said compressible implants can be compressed and then delivered intracorporeally to the mammalian vascular site,
wherein the matrix is compressible and resiliently recoverable.

22. The system of claim 21, wherein the matrix comprises reticulated, interconnected and intercommunicating networks of voids and/or pores to permit ingrowth of tissue.

23. The system of claim 21, wherein the matrix is a polycarbonate polyurethane-urea, polycarbonate polyurea-urethane, polycarbonate polyurethane, or polycarbonate polysiloxane polyurethane.

24. The system of claim 21, wherein the matrix is cross-linked.

25. The system of claim 21, wherein the matrix is thermoplastic.

26. The system of claim 21, wherein the matrix is biocompatible.

27. The system of claim 21, wherein the delivery instrument is a catheter, cannula, needle, syringe, or endoscope.

28. The system of claim 21, which also comprises a loader to compress and introduce the one or more implants into the delivery instrument.

29. The system of claim 21, wherein the delivery instrument has a release member to release the implant or implants at the target site.

30. The system of claim 21, wherein the number of implants is sufficient to occlude the mammalian vascular site.

31. The system of claim 21, wherein the vascular condition is endoleakage.

32. The system of claim 21, wherein the mammalian vascular site is a space between an endovascular graft and a vascular wall.

33. The system of claim 21, wherein the mammalian vascular site is a vessel or vascular defect that needs to be occluded.

34. A method for the treatment or prevention of a vascular condition at a mammalian vascular site, which comprises the step of
delivering one or more reticulated implants in a compressed state to the mammalian vascular site, wherein each implant recovers substantially to its uncompressed state following deployment from a delivery instrument.

35. The method of claim 34, wherein each implant comprises a biodurable, reticulated elastomeric matrix.

36. The method of claim 35, wherein the matrix comprises reticulated, interconnected and intercommunicating networks of voids and/or pores to permit ingrowth of tissue.

37. The method of claim 35, wherein the matrix is a polycarbonate polyurethane-urea, polycarbonate polyurea-urethane, polycarbonate polyurethane, or polycarbonate polysiloxane polyurethane.

38. The method of claim 35, wherein the matrix is cross-linked.

39. The method of claim 35, wherein the matrix is thermoplastic.

40. The method of claim 35, wherein the matrix is compressible and resiliently recoverable.

41. The method of claim 35, wherein the matrix is biocompatible.

42. The method of claim 34, wherein the number of implants is sufficient to occlude the mammalian vascular site.

43. The method of claim 42, wherein from 1 to about 30 implants are delivered.

44. The method of claim 42, wherein the implants are selected so that the total volume of the implants prior to compression and delivery and/or after recovery is from about 60 to about 150 percent of the volume of the target site.

45. The method of claim 44, wherein the implants are selected so that the total volume of the implants prior to compression and delivery and/or after recovery is from about 80 to about 125 percent of the volume of the target site.

46. The method of claim 34, wherein each implant is compressed extracorporeally from a relaxed volume for delivery, the implants are mechanically restrained against expansion during delivery, and each implant is released from the mechanical restraint prior to or during delivery to the mammalian vascular site.

47. The method of claim 34, wherein the implants are delivered through a delivery instrument.

48. The method of claim 47, wherein the delivery instrument is a catheter, cannula, needle, syringe, or endoscope.

49. The method of claim 47, wherein each implant is compressed to have an effective diameter smaller than the effective diameter of the delivery instrument.

50. The method of claim 49, wherein each implant is compressed by a factor of at least 1.1:1.

51. The method of claim 49, wherein each implant is compressed by a factor of at least 2:1.

52. The method of claim 49, wherein each implant is compressed by a factor of up to 4.3:1.

53. The method of claim 49, wherein each implant is compressed by a factor of up to 5.8:1 or higher.

54. The method of claim 34, wherein the vascular condition is endoleakage.

55. The method of claim 34, wherein the mammalian vascular site is a space between an endovascular graft and a vascular wall.

56. The method of claim 55, wherein the vascular site is an aneurysm.

57. The method of claim 56, wherein the aneurysm is an abdominal aortic aneurysm.

58. The method of claim 34, wherein the mammalian vascular site is a vessel or vascular defect that needs to be occluded.

59. A method for the treatment or prevention of a vascular condition at a mammalian vascular site, which comprises:
compressing one or more implants to a dimension suitable to be loaded into a delivery instrument,
loading the compressed implant or implants into the delivery instrument,
tracking the loaded delivery instrument through an introducer or guide sheath to a target site, and
releasing the compressed implant or implants at the target site.

60. The method of claim 59, wherein the matrix is a polycarbonate polyurethane-urea, polycarbonate polyurea-urethane, polycarbonate polyurethane, or polycarbonate polysiloxane polyurethane.

61. The method of claim 59, wherein the matrix is cross-linked.

62. The method of claim 59, wherein the matrix is thermoplastic.
